(19) United States
(12) Patent Application Publication
(10) Pub. No.: US 2005/0261940 A1
(43) Pub. Date: Nov. 24, 2005

(54) Method and apparatus for managing drug inventory at point of care

(76) Inventors: James Alvin Gay, Newmarket (CA); Brett Alexander Holtby, Aurora (CA); William Keith Thomas, Toronto (CA)

Correspondence Address: SMART & BIGGAR/FETHERSTONHAUGH & CO.;P.O. BOX 2999, STATION D, 900-55 METCALFE STREET, OTTAWA, ON, K1P5Y6, CA

(21) Appl. No.: 10/848,157

(22) Filed: May 19, 2004

(51) Int. Cl. G06F 17/60

(52) U.S. Cl. 705/3; 705/28

(57) ABSTRACT

A system for managing administration of prescription drugs supplied by a medication supplier to a health care facility, comprising a central application server site for maintaining Medical Administration Records (MARs) pertaining to residents in the health care facility and having data communication paths to the medication supplier and the health care facility, and a plurality of portable devices for use in the health care facility for accessing and displaying the MARs at point of care of the residents and initiating order of the prescription drugs from the pharmacy via the central server site.

### FIELD OF INVENTION

The invention relates generally to computerized patient care systems, and more particularly to a method and apparatus for managing drug administration, treatment, procedures and care delivery administration.

### BACKGROUND OF THE INVENTION

It is a well established procedure in hospitals, chronic or long term health care facilities, etc. to manage the administration of drug inventory by maintaining a Medication Administration Record (MAR) for each patient (the terms “patient” and “resident” are used interchangeably in this specification). The MAR is typically in the form of a patient chart that lists all of the medications for a particular patient along with the dosages and times of administration by hospital staff such as nurses and doctors. Upon administering a dosage of medication, the hospital staff member initials an appropriate entry on the MAR to indicate the time of administration. Once a medication has been depleted, separate forms must be used to order a refill prescription. The MAR form also often includes special notices relating to allergies, or other data relevant to the resident and medications.

The current process results in up to 20% of drugs being given in error or omitted, and consequent high liability insurance costs. Drugs that are given in error or omitted may lead to hospitalization or lawsuits and the current process does not provide a good audit trail for regulators or litigation defense. The current process also results in noticeable materials, mailing and documentation costs as well as potential drug wastage. Pharmacists and physicians have noticeable time wasted on order entry, transcription, re-entry and clarification as pharmacies may not receive legible orders for replenishment.

In addition to the evident costly inefficiencies of hand-entering information to the MAR, completion of the forms is also prone to human error.

A number of computerized systems are known for drug management in hospitals:

U.S. Pat. No. 6,181,979 discloses a drug preparation system for indicating which of a plurality of drug processing or inspection stations are busy so that drugs can be prepared in an optimum way. Patient data recorded on prescriptions received at a pharmacy reception is entered into a host computer through an input device. On command of the host computer, patient data are transferred to respective drug processing units. Each processing unit enters time data including drug preparation completion time into trays with a memory function for collecting drugs prepared. The drugs collected in each tray are inspected and sent to a drug pickup window, where the time data are transferred to a data processing unit. Based on the transferred data, a total processing time in each station is displayed on a display.

U.S. Pat. No. 6,088,695 discloses a system and method for coding medical data generated by a plurality of different care providers for use by a primary care provider. The medical data is coded in a uniform manner, such as a bar code, and is easily transported and stored, both in physical and electronic form.

U.S. Pat. No. 6,032,155 sets forth a method and apparatus for administering prescribed medication to a patient. The prescribed medication administration system and apparatus dispense prescribed medication via a portable container that is connected to a workstation. The workstation activates the portable administration device only after verifying that the medication is being given to the correct patient by an authorized healthcare worker. An information device is secured to the portable container during transport and administration of the medication to the intended patient. The information device prevents access to the medication or warns the healthcare worker of a potential error if the medication is delivered to the wrong patient or administered by an unauthorized healthcare worker. The information device records actual consumption information, and delivers this information back the workstation database or to a hospital or pharmacy database.

U.S. Pat. No. 6,032,119 sets forth a system for personalized display of patient health care information directly on a customized image of a body. The patient's medical record, standards of care for the condition, prescribed treatments, and patient input are applied to a generalized health model of a disease to generate a personalized health model of the patient. The personalized health model comprises an HTML file encoding an image map of a body. The body image illustrates the health condition of the individual patient.

U.S. Pat. No. 6,021,392 discloses a system in which a drug distribution center operates a computer software drug inventory management program in electronic communication with a health care provider computer software program for drug and health care supply distribution to patients. The system incorporates low unit dose measure drug and supply packaging including bar codes for automatically tracking drug information. The system further includes hand held drug information collection units for collecting the drug and supply information from the bar coded packages. The system provides complete drug and supply tracking from the drug supplier to a nursing station automated drug/supply dispensing machine.

U.S. Pat. No. 5,924,074 discloses a medical records system that creates and maintains all patient data electronically. The system captures patient data, such as patient complaints, lab orders, medications, diagnoses, and procedures, at its source at the time of entry using a graphical user interface having touch screens. Using pen-based portable computers with wireless connections to a computer network, authorized healthcare providers can access, analyze, update and electronically annotate patient data even while other providers are using the same patient record. The system includes the capability to access reference databases for consultation regarding allergies, medication interactions and practice guidelines.

U.S. Pat. No. 5,842,976 provides a method for tracking drug inventory distribution to patients, using one to one distributed links between independent computers. The method for tracking inventory is distributed, and allows only one-way flow of information. The method individually links points between medication and medical product suppliers with the users at the facilities. The system contemplates a system linking discrete facilities with their discrete suppliers of products on a point-to-point basis.

### SUMMARY OF THE INVENTION

According to one broad aspect, the invention provides a system for managing administration of medications supplied by a medication supplier to a healthcare facility comprising: a central application server site for maintaining medical administration records pertaining to residents in said healthcare facility and having a first data communication path to said medication supplier and a second data communication path to each healthcare facility; and a plurality of portable devices for use in the healthcare facility for accessing and displaying said MARs at healthcare of said residents and initiating order of said medications from said medication supplier via said central server site.

In some embodiments, each medical administration record comprises at least an identification of any medications to be given, and when.

In some embodiments, the second data communication path is implemented using a VPN (virtual private network).

In some embodiments, a system is adapted to manage administration of medications to healthcare facilities which are long term care facilities.

According to another broad aspect, the invention provides a system for managing administration of medications supplied by a medication supplier to a healthcare facility comprising: a central application server site for maintaining medical administration records pertaining to residents in said healthcare facility and having a first data communication path to said medication supplier and a second data communication path to each health care facility; and a computer readable medium having a first application stored thereon adapted for use on portable devices for use in the health care facility for accessing and displaying said medical administration records at point of care of said residents and initiating order of medicatons from said medication supplier via said central server site.

In some embodiments, the first application is delivered by the application server site as a series of downloadable pages to the portable devices.

In some embodiments, a system further comprises a computer readable medium having an administrative and reporting application stored thereon for managing the creation and amendment of the medical administration records.

In some embodiments, the administrative and reporting application is delivered by the application server site as a series of downloadable pages to devices in the healthcare facilities.

In some embodiments, a system for managing administration of medications supplied by a plurality of medication suppliers to a plurality of healthcare facilities wherein: the central application server site is adapted to maintain medical administration records pertaining to residents in each healthcare facility and has a respective first data communication path to each said medication supplier and a respective second data communication path to each healthcare facility; and the system further comprising a plurality of portable devices for use in each health care facility for accessing and displaying said medical administration records at point of care of said residents and initiating order of said medications from an appropriate one of said medication suppliers via said central application server site.

In some embodiments, a system for managing administration of medications, treatments, procedures and care delivery to a plurality of health care facilities by a plurality of external providers comprising at least said medication supplier wherein: the central application server site is adapted to maintain medical administration records pertaining to residents in each health care facility and has a respective first data communication path to each said external provider and a respective second data communication path to each health care facility; and the system further comprising a plurality of portable devices for use in each health care facility for accessing and displaying said medical administration records at point of care of said residents and initiating order of said medications or services from an appropriate one of said external providers via said central application server site.

In some embodiments, each said first path is secure, and each said second path is secure.

In some embodiments, a system further comprises at least one device in each healthcare facility adapted to run an administrative and reporting application for managing creation and amendment of the medical administration records.

In some embodiments, the portable devices in a given health care facility are configured in a wireless LAN.

In some embodiments, each said second data communication path between said central server site and said health care facility is implemented using a VPN, and wherein said pharmacy incorporates gateway software to contact said server site for uploading and downloading prescription information via staging tables.

In some embodiments, a system is adapted to, for each healthcare facility: maintain an association between the point of care facility and a respective medication supplier of said plurality of external providers; receive medication orders from the first applications of the healthcare facility; and send orders to the respective medication supplier.

In some embodiments, a system is adapted to, for each healthcare facility, and for each of at least one resident in the point of care facility: maintain an association between the patient and a respective medication supplier of said plurality of external providers; receive medication orders from the first applications for the patient; and send medication orders to the respective medication supplier.

In some embodiments, upon request from one of said portable devices, the central server site generates a list of medical administration records for a selected time and downloads the list of medical administration records to the portable device, the list of medical administration records including portions of medical administration records of any residents of a selected set of residents that are scheduled to receive medication or treatment at the selected time, the portions of medical administration records including at least any medications or treatments to be administered at the selected time.

In some embodiments, each downloaded medical administration record comprises: an identification of any medications to be administered at the selected time; an identification of any procedures to be performed at the selected time; allergy information; any other comments; and a digital photograph.

In some embodiments, the portable devices are adapted to generate a display pertaining to a selected resident containing the medications, procedures, allergy information, digital photograph and any other comments.

In some embodiments, a system is further adapted to receive a user input upon retrieval of a medication and upon administration of a medication, and to display each medication in a first manner initially, in a second manner visually distinct from said first manner after the medication has been retrieved, and in a third manner visually distinct from said first and second manners after the medication has been administered.

In some embodiments, a system is further adapted to provide a user input at the portable device for selecting reordering of a given medication; wherein responsive to said user input at the portable device, the application server site generates an order for the medication and sends this automatically to an appropriate medication provider.

In some embodiments, a system is further adapted to provide a user input at the portable device for selecting reordering of a given medication, treatment, procedure or care delivery; wherein responsive to said user input at the portable device, the application server site generates an order for the medication, treatment, procedure or care delivery and sends this automatically to an external provider.

In some embodiments, a system is further adapted to display that a given medication has been reordered until the medication has been received.

In some embodiments, a system is further adapted to store vital sign information for residents.

In some embodiments, a system comprises a user interface on the portable devices for entering vital sign information.

In some embodiments, a system is further adapted to generate a request to take vital signs prior to administering a given medication.

In some embodiments, a system is adapted to display a list of resident identifiers of residents to receive medication, and to display each resident's identifier in a different manner after completing a med pass for the resident.

In some embodiments, displaying a resident's identifier in a different manner after completing a med pass for the resident comprises listing them on a different display page on the portable device.

In some embodiments, a is further adapted to present a plurality of options on the portable device for each medication to indicate whether the medication was successfully administered or not, and if not to enter one of a plurality of reason codes.

In some embodiments, the reason codes are customizable on a per healthcare facility basis.

In some embodiments, a system is adapted to display residents who are on leave of absence so that a verification that they are in fact still out of the facility can be performed.

In some embodiments, each said medical administration record further comprises an identification of any as needed medications for the associated resident, the system being further adapted to track administration of any as needed medications.

In some embodiments, each said medical administration record further comprises an identification of any as needed medications for the associated resident, the system being further adapted to track administration of any as needed medications; wherein the system is further adapted to initially display as needed medications in a manner visually distinct from said first manner.

In some embodiments, a system is further adapted to track a reason for administration of an as needed medication.

In some embodiments, a system is further adapted to maintain a history of administration of an as needed medication.

In some embodiments, a system is further adapted to automatically include a medical administration record of a resident who was administered an as needed medication in a list of medical administration records generated at a subsequent time and request effectiveness information from a health care worker in respect of how effective the as needed medication was.

In some embodiments, a system is further adapted to include a resident with no scheduled medications or treatments in a list of medical administration records for a selected time if they have as needed medications.

In some embodiments, a system is adapted to include a resident with no scheduled medications or treatments in a list of medical administration records for a selected time if they have as needed medications in a manner visually distinct from residents with scheduled medications or treatments.

In some embodiments, a system is further adapted to track standing orders for each of a plurality of health care providers.

In some embodiments, a system is further adapted to download for each resident the standing order information pertaining to an associated one of said healthcare providers.

In some embodiments, each said medical administration record further comprises an identification of standing order medications for the associated resident, the system being further adapted to track administration of any standing order medications.

In some embodiments, each said medical administration record further comprises an identification of standing order medications for the associated resident, the system being further adapted to track administration of any standing order medications; wherein the system is further adapted to initially display standing order medications in a manner visually distinct from said first manner.

In some embodiments, a system provides a user selection of whether or not to display standing order medications for the associated resident.

In some embodiments, a system is further adapted to track a reason for administration of a standing order medication.

In some embodiments, a system is further adapted to maintain a history of administration of a standing order medication.

In some embodiments, a system is further adapted to automatically include a medical administration record of a resident who was administered a standing order medication in a list of medical administration records generated at a subsequent time and request effectiveness information from a health care worker in respect of how effective the standing order medication was.

In some embodiments, a system is further adapted to include a resident with no scheduled medications or treatments in a list of medical administration records for a selected time if there are standing order medications.

In some embodiments, a system is further adapted to: after completing an attempt at administering medications to or treatments for a given resident wherein a failure to administer one or more medications or treatments has occurred, the system is adapted to keep the resident's medical administration record on the list of medical administration records to allow a re-visit during a current med pass.

In some embodiments, a system is adapted to request a reason code from a health care worker following failure to administer a medication.

In some embodiments, a system is adapted to allow a re-visit only for failures with particular reason codes.

In some embodiments, a system is adapted to display a resident's identifier in a visually distinct manner if they are to receive a revisit.

In some embodiments, said portable devices incorporate a user log-in function to restrict access to authorized persons.

In some embodiments, said portable devices include a user interface for displaying selected ones of said medical administration records according to location, time and date within said health care facility.

In some embodiments, said selected ones of said medical administration records are accessed by database replication between said portable devices and said central server site.

In some embodiments, said portable devices include a user interface for displaying sites for injection administration of said drugs to said residents.

In some embodiments, the system further comprises functions for receiving medication orders enabling comparison of a received medication and a medication order and for entering follow-up notations based on any discrepancies therebetween.

In some embodiments, the system is adapted to: upon user request, download outstanding medication orders to one of said portable devices; upon scanning of a received medication, generate a display containing information pertaining to the scanned medication, and information pertaining to a medication order for the particular received medication so that discrepancies can be identified.

In some embodiments, a system further comprises: at least one function for reporting destruction of medications.

In some embodiments, a system comprises: a user interface on the portable device for entering an identifier of a medication to be destroyed, and for entering at least one count of the medication to be destroyed.

In some embodiments, the application server site collects information pertaining to the type and number of medication destroyed and generates a report upon request for use in obtaining credit for the destroyed medications.

In some embodiments, a system further comprises at least one device executing an administrative and reporting application for creating and updating said medical administration records and initiating physician's orders of said medications.

In some embodiments, the at least one device comprises at least one workstation.

In some embodiments, the administrative and reporting application runs on the portable devices.

In some embodiments, said administrative and reporting application incorporates a user log-in function to restrict access to authorized persons.

In some embodiments, said administrative and reporting application includes a user interface for uploading resident photographs to said central server site.

In some embodiments, said administrative and reporting application includes a user interface for updating status of said residents.

In some embodiments, the administrative and reporting application comprises a plurality of screens downloadable by the application server site.

In some embodiments, each physicians order comprises: an identity of a medication and an identity of the resident the medication is being ordered for; time of administration information; and frequency of administration information.

In some embodiments, the system is adapted to generate an order for the medication, and to enter the medication on the resident's medical administration record.

In some embodiments, the system is adapted to present a list of all medications or treatments for a selected resident, and to allow amendment of time or frequency information, and to allow discontinuing a medication.

In some embodiments, a system further comprises: a user interface screen(s) for indicating that a particular resident is going on leave of absence.

In some embodiments, the user interface screen(s) for indicating that a particular resident is going on leave of absence lists all of the medications being administered to the resident and collect “doses out” information identifying numbers of doses of each medication being taken out of the facility.

In some embodiments, the user interface screen(s) for indicating that a particular resident is going on leave of absence include an input for indicating the resident has returned, and list all of the medications being administered to the resident and collect “doses in” information identifying numbers of doses of each medication being returned to the facility.

In some embodiments, a system is further adapted to compute a difference between the doses out and the doses in and to determine whether a proper amount of medication was administered for an amount of time the particular resident was on leave of absence.

In some embodiments, the administrative and reporting application is adapted to collect at least one of: allergy information, medical condition information, and any other comments, and lab test requirements.

In some embodiments, the administrative and reporting application is adapted to collect responsible party information pertaining to individuals who are to be allowed to leave the facility with a given resident.

In some embodiments, the administrative and reporting application is adapted to collect drug plan information for each resident and to include drug plan information in orders for medication made for the resident.

In some embodiments, the system is adapted to track a health care facility inventory of at least one of the medications. The system may be further adapted to track administration of each of the at least one of said medications to said residents and to update the health care facility inventory of the at least one of the medications.

Another embodiment provides software on a computer readable medium for implementing any feature or combination of features summarized above.

For example, another broad aspect provides a computer readable medium having executable instructions stored thereon for managing administration of medications supplied by a medication supplier to a health care facility, the executable instructions comprising code for maintaining medical administration records pertaining to residents in said health care facility and for communicating via a first data communication path to said medication supplier and via a second data communication path to each health care facility; and code for use on a plurality of portable devices for use in the health care facility for accessing and displaying said medical administration records at point of care of said residents and initiating order of said medications from said medication supplier via said central server site.

In some embodiments, the computer readable medium is adapted to manage administration of medications supplied by a plurality of medication suppliers to a plurality of health care facilities, and the instructions further comprise code for maintaining medical administration records pertaining to residents in each health care facility and communicating via a respective first data communication path to each said medication supplier and via a respective second data communication path to each health care facility; and code for use on portable devices for use in each health care facility for accessing and displaying said medical administration records at point of care of said residents and initiating order of said medications from an appropriate one of said medication suppliers via said central application server site.

In some embodiments, the computer readable medium is for managing administration of medications, treatments, procedures and care delivery to a plurality of health care facilities by a plurality of external providers comprising at least said medication supplier, and the instructions comprise code for maintaining medical administration records pertaining to residents in each health care facility and communicating via a respective first data communication path to each said external provider and via a respective second data communication path to each health care facility; and code for use on portable devices in each health care facility for accessing and displaying said medical administration records at point of care of said residents and initiating order of said medications or services from an appropriate one of said external providers via said central application server site.

In some embodiments, the instructions further comprise code for an administrative and reporting application for managing creation and amendment of the medical administration records.

### BRIEF DESCRIPTION OF THE DRAWINGS

Preferred embodiments of the invention will now be described with reference to the attached drawings, in which:

FIG. 1 is a block diagram of the computerized patient care system according to an embodiment of the present invention;

FIG. 2 is a block diagram of a wireless LAN of portable devices forming part of an example implementation of the patient care system of FIG. 1;

FIG. 3 is a graphical user interface showing user login to a portable device as shown in FIG. 2;

FIG. 4 is a graphical user interface showing three displayed system options following user login;

FIG. 5 is graphical user interface showing user entry of location, date and time of a med pass;

FIG. 6 is a graphical user interface to alert the user that data is being synchronized within the portable device;

FIG. 7 is graphical user interface listing names of residents in connection with which the med pass is to be performed;

FIG. 8 is a graphical user interface of a selected patient's MAR, with allergy alert window, for the chosen hour and date of the med pass;

FIG. 9A is a graphical user interface of a selected patient's MAR, after having cleared the allergy alert window;

FIG. 9B is a graphical user interface of a selected patient's MAR, including a procedure;

FIG. 10 is graphical user interface showing a screen for entering the patient's vitals;

FIG. 11 is a graphical user interface of the selected patient's MAR, after having retrieved the various medications;

FIG. 12 is a graphical user interface of patient sites for medication injection;

FIG. 13A is a graphical user interface for entering dosages administered to a patient on an as needed basis;

FIG. 13B is a graphical user interface showing a patient requiring a “follow-up” visitation subsequent to an administration of an as needed medication;

FIG. 14 is a graphical user interface for selecting action on the effectiveness of previously administering medication to the patient;

FIG. 15 is a graphical user interface showing the various choices a nurse has when administering a medication or treatment;

FIG. 16 is a graphical user interface of the selected patient's MAR, after having administered the various medications;

FIG. 17A is a graphical user interface similar to FIG. 7, but with the patient to whom medications have been administered being removed from the list and the next patient being indicated as being on a leave of absence;

FIG. 17B is a graphical user interface for confirming whether a resident is on leave of absence;

FIG. 18 is a graphical user interface for receiving med orders from a pharmacy;

FIG. 19 is a graphical user interface for destruction of discontinued medications;

FIG. 20 is a graphical user interface showing user login to a administrative and reporting application provided by an embodiment of the present invention;

FIG. 21 is a graphical user interface showing a resident search using the administrative and reporting application;

FIG. 22 is a graphical user interface showing the results of the search;

FIG. 23 is a graphical user interface listing all active medications or treatments for a resident; this is also a graphical user interface for discontinuing medications via the administrative and reporting application;

FIG. 24 is a graphical user interface for entering new orders for medications or treatments via the administrative and reporting application;

FIG. 25A is a graphical user interface for recording a resident's leave of absence check-out via the administrative and reporting application;

FIG. 25B is a graphical user interface for recording a resident's leave of absence check-in via the administrative and reporting application;

FIG. 26 is a graphical user interface for recording a resident's file details via the administrative and reporting application;

FIG. 27 is a graphical user interface for recording a resident's allergies and notes via the administrative and reporting application;

FIG. 28 is a graphical user interface for recording a resident's responsible parties via the administrative and reporting application;

FIG. 29 is a graphical user interface for recording a resident's drug plans via the administrative and reporting application;

FIG. 30 is a graphical user interface for a first step of recording admission of a new resident via the administrative and reporting application; and

FIG. 31 is a graphical user interface for a second step of recording admission of a new resident via the administrative and reporting application.

### DETAILED DESCRIPTION OF THE PREFERRED EMBODIMENT

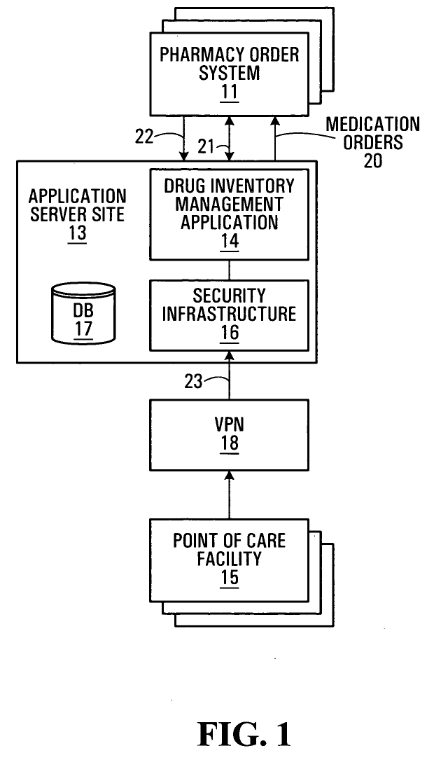

FIG. 1 is a block diagram of a computerized patient care system provided by an embodiment of the invention. The system consists of a centrally hosted application server site 13 running a drug inventory management application 14 provided by another embodiment of the invention. The centrally hosted application server site preferably has a security infrastructure 16. The application server site 13 is connected to a point of care facility 15 as indicated by connection 23. The application server site 13 also interfaces with a pharmacy 11 over connection 21. In preferred embodiments, the application server site is capable of interfacing with multiple pharmacies 11 over respective connections with three such pharmacies illustrated in the figure. Similarly, preferably the application server site 13 is capable of interfacing with multiple point of care facilities 15 over respective connections with three such facilities shown in FIG. 1. While pharmacies are referred to throughout, more generally any medication supplier may be involved to supply the medications.

Connection Between Point of Facility and Application Server Site

In the illustrated example, connection 23 is realized with a VPN 18 which might for example be established over the public internet. Preferably, the application server site consists of standard server hardware capable of setting up VPN (virtual private network) connections such as the connection 18 to the point of care facility 15 of FIG. 1. Preferably, the security infrastructure 16 sets up a secure VPN with encryption such that data transmitted over the public internet to the point of care facility is secure. While in preferred embodiments the communications between the application server site 13 and the point of care facility is done using VPNs over the public internet, if there is enough traffic between the application server site 13 and a given point of care facility, it might be practical to implement a dedicated link. More generally, connection 23 may be realized using any appropriate communications links between the application server site 13 and the point of care facility 15, for example by ISDN, cable, T1 or any other conventional means.

As discussed above, preferably data security is provided for communications between the applications server site 13 and point of care facility 15. To that end, a VPN may be provided via Internet Service Providers (ISPs) that support PPTP. Another embodiment incorporates SSL technology without the VPN.

Connection Between the Application Server Site and Pharmacy

The connection 21 between the application server site 13 and the pharmacy 11 is preferably also conducted over the Internet. However, direct connections may also be implemented. The application server site 13 preferably communicates with the pharmacy system 11 in a manner that is consistent with the pharmacy system computer protocol. Where the pharmacy has inadequate computer communication capabilities, the prescription orders can be communicated via fax, or manually communicated. In some embodiments, the pharmacy system is provided with a VPN capability and communication between the pharmacy system 11 and application server site 13 is done using a private VPN over the public internet. More generally, any appropriate means of communicating between the pharmacy 11 and the point of care facility 15 may be employed. For example, in pharmacies that use the industry-standard Kroll and Tech Rx (Propharm) systems, gateway software may be provided on the pharmacy systems 11 to dial up the server site 13 for uploading and downloading prescription information by writing to staging tables.

Application Server Site

The Application Server Site 13 is adapted to establish connections 21,23 to the pharmacy 11 and point of care facility 15 as described above. Preferably, the application server site 13 is adapted to interact with existing pharmacy software forming part of the pharmacy order system 11. The application server site 13 sends medication orders 20 to the pharmacy 11. The pharmacy 11 provides data 22 in return. This data 22 is prescription data which might for example consist of resident information, medication directions, bar code information and hours of administration.

The application server site 13 is preferably provided independent of the point of care facility, and provides an interface between the point of care facilities 15 and the pharmacy 11 for multiple point of care facilities in multiple pharmacies. However, in some embodiments the application server site may be co-located with either one of the pharmacies or one of the point of care facilities.

The application server site 13 typically also features a database 17 containing data for use by the drug inventory management application 14 as described in detail below. Preferably, the application server site 13 is a SQL server, for example Microsoft SQL Server 2000 Enterprise Edition. Of course other suitable server designs may alternatively be employed.

Point of Care Facility

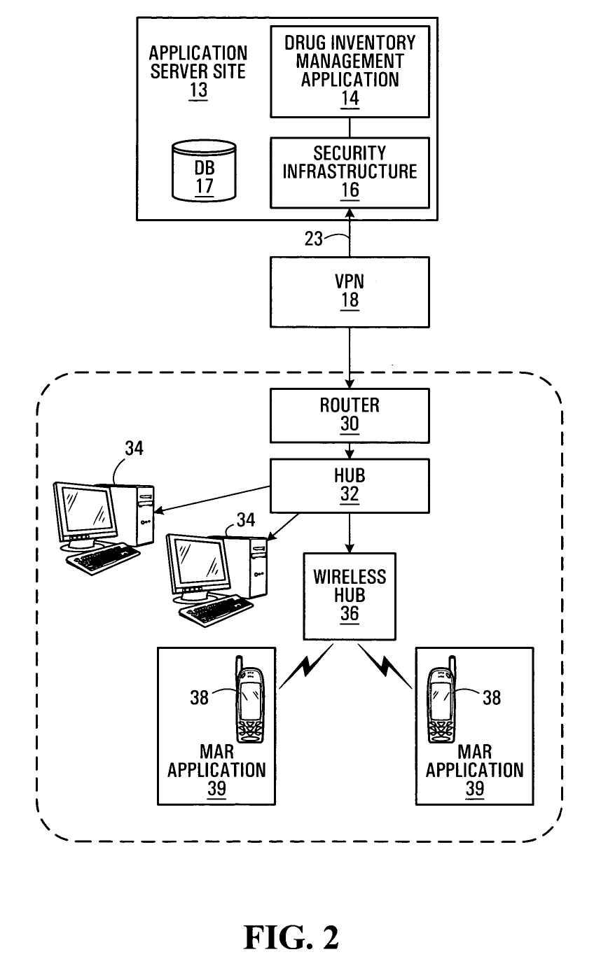

The point of care facility 15 is preferably a long term care facility, a nursing facility or a hospital. Further details of an example of infrastructure which might be set up in the point of care facility 15 will now be described with reference to FIG. 2.

A router 30 or other suitable network element provides access via connection 23 to the application server site 13 as described above. A network hub 32 is also shown which connects to one or more other wireless hubs, a single one of those hubs 36 being shown for the ease of illustration. Two portable wireless devices 38 equipped to run a MAR application 39 provided by an embodiment of the invention are shown communicating wirelessly with the wireless hub 36. Also shown are two workstations 34 connected to the hub 32 each equipped to run a administrative and reporting application provided by another embodiment of the invention.

The network configuration shown in FIG. 2 is simply an example. The important feature is that portable devices 38 equipped with MAR applications 39 are able to communicate with the application server site 13. In the illustrated example, this is achieved by the wireless hub 36, the network hub 32, the router 30, VPN 18. However, other routes may alternatively be employed. The portable devices 38 may be any suitable devices capable of running a MAR application 39 as detailed below.

Portable Devices

Nurses or other authorized healthcare providers use the wireless portable devices 38 to access patient medical records as described in greater detail below Preferably, the portable devices 38 use an operating system, which supports application development using standard languages (e.g. VB, C++, Java). Data may be “pulled” from the server site 13 to a portable device 38 in response to being selected, for example, via a SQL statement which creates and populates a local table. Data may also be “pushed” from the portable device 38 to the server site 13 in a similar manner. Preferably, each portable device 38 incorporates a bar code scanner for user authorisation and patient verification. The illustrated example shows two portable devices 38. More generally, any appropriate number can be deployed in a given point of care facility 15. In some embodiments, the portable devices provide seamless support of VPN, as discussed above with reference to the security infrastructure.

Preferably, the portable devices 38 are configured in a wireless LAN and communicate MAR data and medication orders to the application server site 13 over the internet using encryption or other security measures.

The wireless LAN preferably has adequate speed and range, and is capable of accommodating a plurality of simultaneous users, and features which might for example include roaming support, power off of Ethernet, user authentication and 128 bit encryption (WEP). To that end, suitable wireless LANs such as manufactured by Lucent, 3COM, Proxim, Linksys and D-Link may be used in some examples to implement the system according to the present invention.

Workstations

The workstations 34 run the above-introduced administrative and reporting application for administration functions, as discussed in greater detail below.

MAR Application

The MAR application 39 running on the portable devices 38 will now be described in detail with reference to a number of graphical user interface displays. It is to be clearly understood that these graphical user interface displays are provided for the purpose of understanding the invention, but that not all details of every display need to be implemented in a given embodiment. In describing the graphical user interface displays, enough detail will be provided to enable one skilled in the art to make and use the invention by considering the description in combination with the graphical user interface displays.

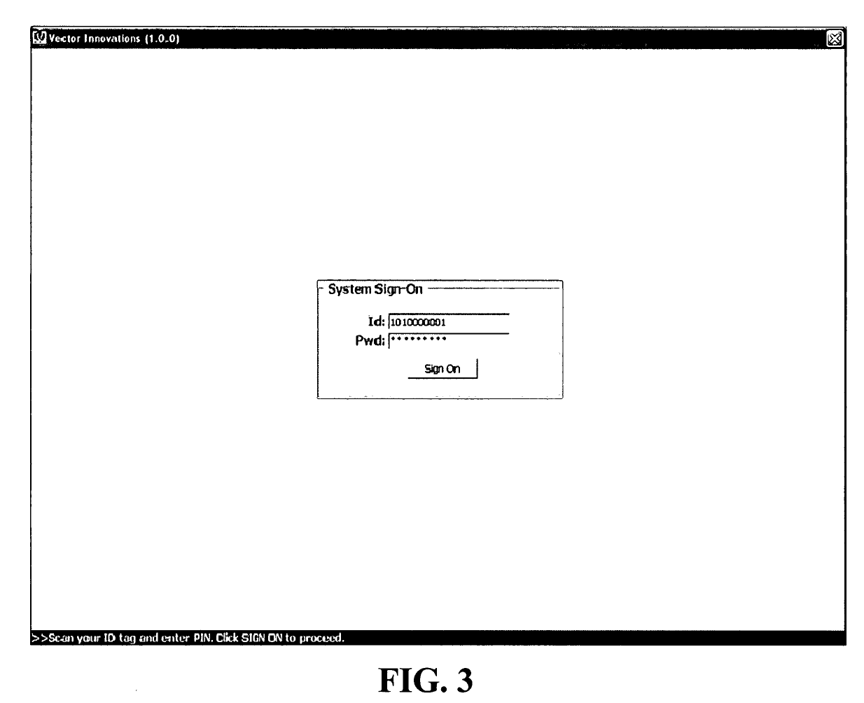

To begin, preferably the MAR application requires a user of a portable device to log into the system so as to prevent unauthorized access. An example of a system sign on graphical user interface display is shown in FIG. 3. In this particular example, the user is required to enter a user ID and PIN (personal identification number). This information is passed up through the network to the application server site 13 where user verification takes place, and access is granted or not. Other methods of verification that do not require the user to enter a user ID and PIN may alternatively be employed, for example the use of an ID card which can be swiped by the device with a bar code reader, or by using a biometric device.

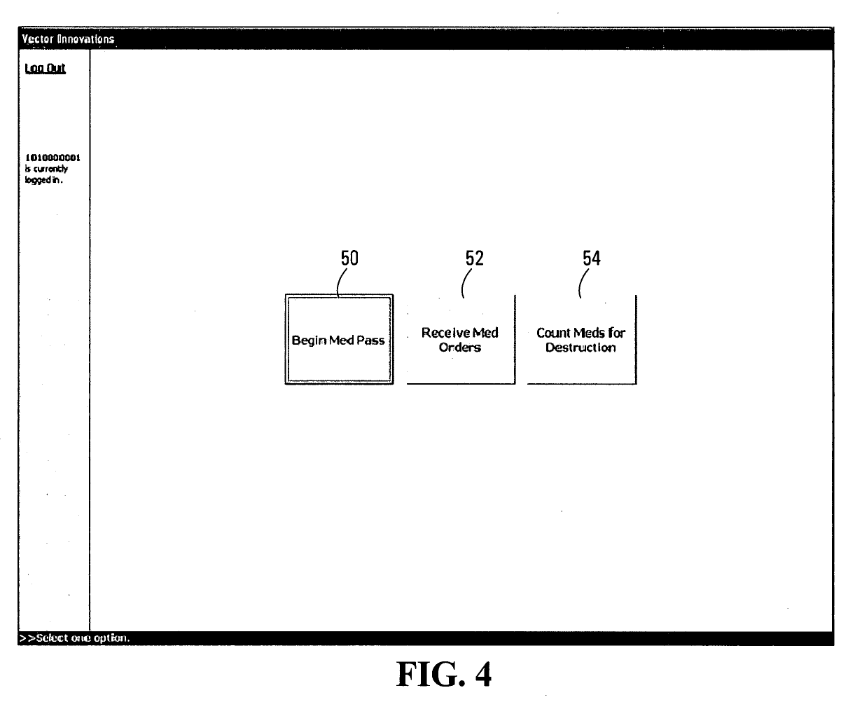

FIG. 4 is an example of a graphical user interface display which might be displayed after a successful system sign on. This presents a user with three options: begin med pass 50, receive med orders 52 and count medications for destruction 54. Preferably all of these three functions are implemented in the MAR application. However, some embodiments may only include one or two of these functions. Each of these functions will now be described in detail. A user of the portable device is capable of selecting any of the three functions using an appropriate user interface on the device. This might for example involve tabbing a cursor, or controlling a mouse or keyboard. Any appropriate mechanism of selecting one of the three functions is contemplated.

Begin Med Pass

The “begin med pass” option starts the portion of the MAR application responsible for coordinating a med pass. A med pass is the process of a health care provider passing through the point of care facility and administering medication to patients.

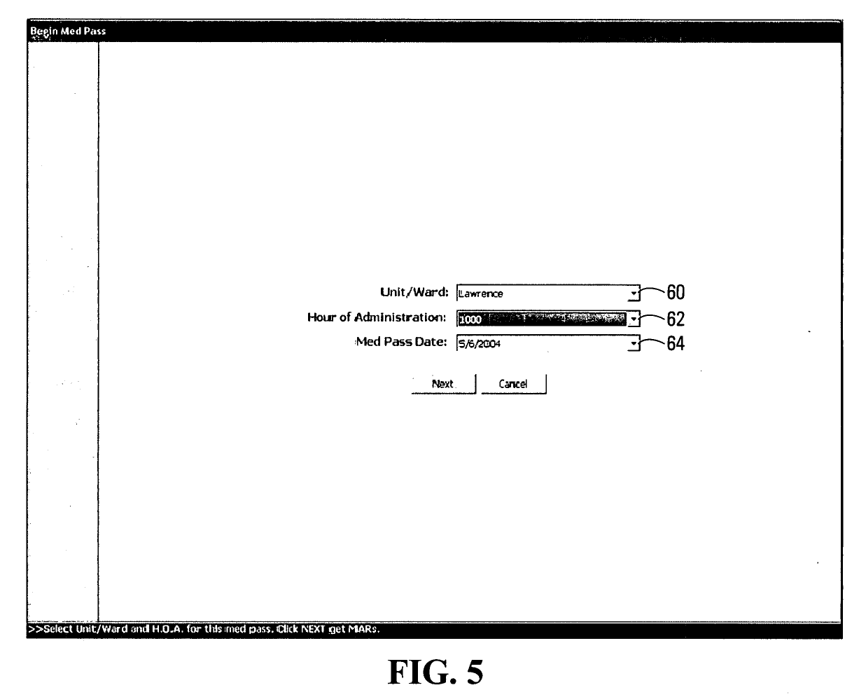

Upon selecting the Begin Med Pass option, the user is presented with a screen for inputting the location within the facility 15 at which the med pass (medical pass through facility) is to be performed as well as the hour and date of its performance. An example of a graphical user interface display for achieving this is shown in FIG. 5. This display allows a user of the device to select from a plurality of unit/wards 60, a plurality of possible hours for administration 62, and a date for conducting the med pass 64. Preferably, these are made available to the user via a series of pull down menus. The MAR application interfaces with the application server site 13 to obtain the information necessary to populate these pull down tables. Preferably, the information used to populate the tables is selected in accordance with the identification of the person who logged on to the system. This information is downloaded to the MAR application when the user logs on to the system.

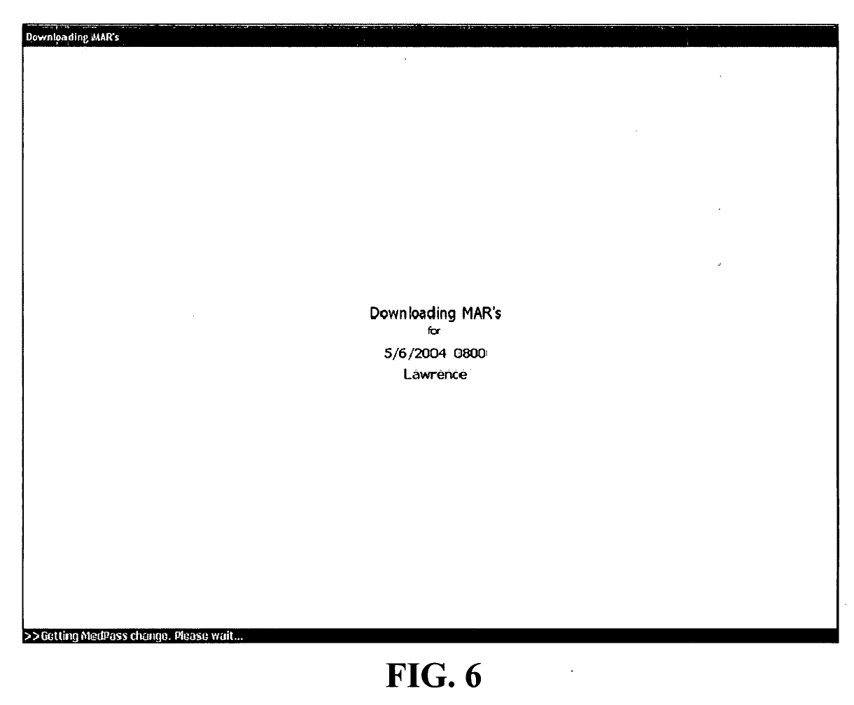

FIG. 6 shows an example of a screen which might be presented to a user on the portable device while the MAR application is synchronizing the MARs it has stored for patients with the data on the server site 13. In the illustrated example, FIG. 6 shows the message “Downloading MARs for May 6, 2004 08:00 Lawrence” since this is the unit/ward and are administration selected by the healthcare worker.

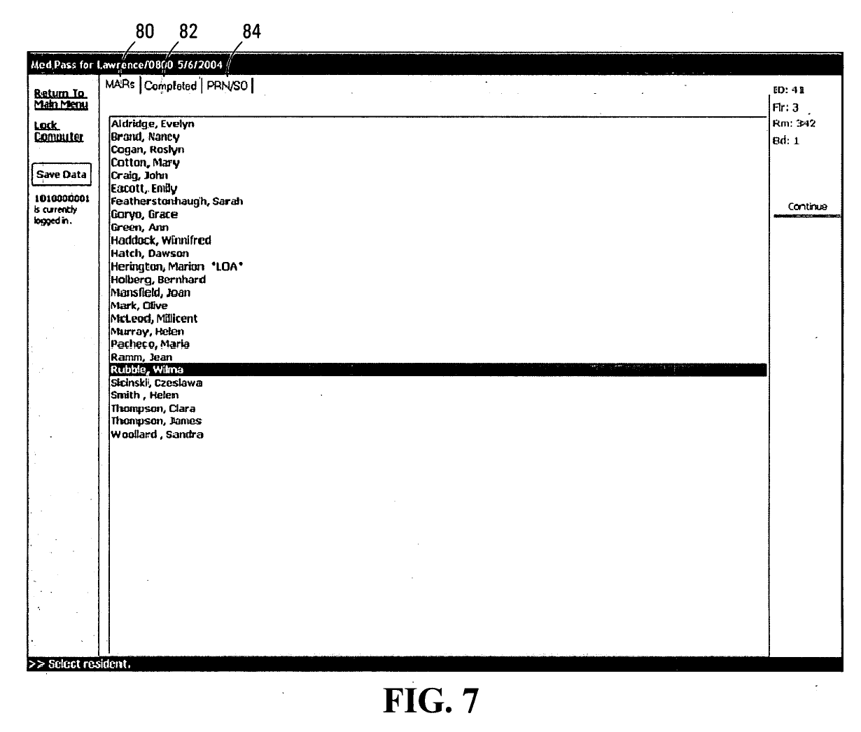

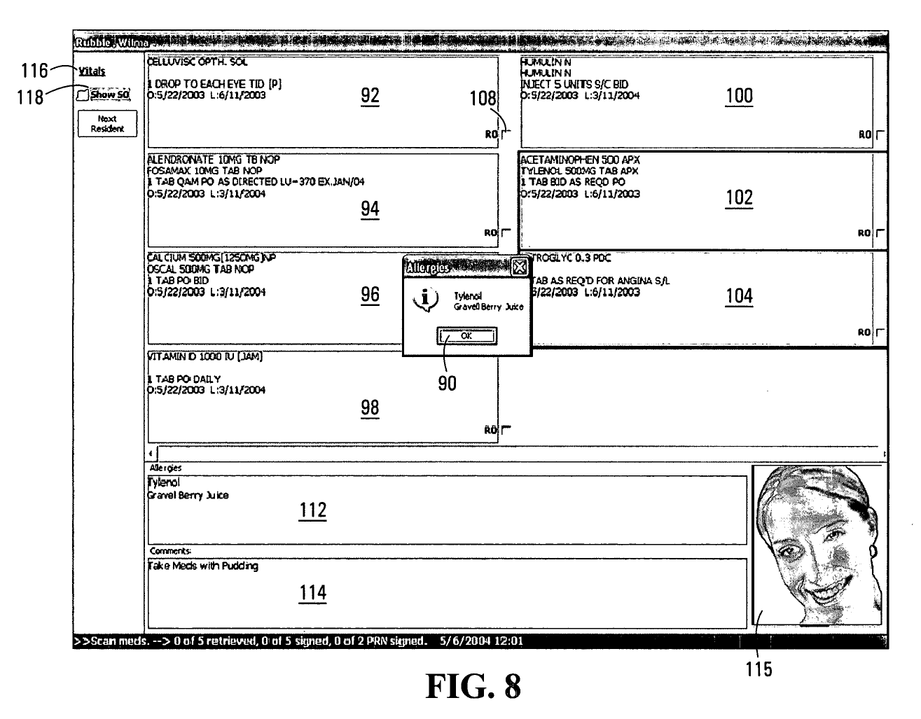

The user is then provided with a screen that lists the residents (patients) in connection with which the med pass is to be performed. The screen has three high level tabs 80 for MARs, 82 completed, and 84 PRN/SO for as needed medications, and standing orders. The display of FIG. 7 is that associated with the MARs tab 80. A particular patient's MAR can be selected from the list that is displayed in the example of FIG. 7, and a graphical display of the selected patient's MAR is presented for example such as shown in FIG. 8 for a particular patient. Furthermore, for the list of patients of FIG. 7, only the patients that require medication for the particular time will be listed in that display. Once a med pass has been completed for a particular patient, that patient will disappear from the display when the MARs tab 80 is selected in FIG. 7, and will be listed under the display that would be presented when the completed tab 82 is selected. A patient of the selected ward who is not included in a given med pass, will be listed under the PRN/SO tab 84. Patients names listed in the display of FIG. 7 may have an additional indicator beside their names. The indicator “LOA” means that the patient is on leave of absence. For a patient with the LOA indicator, the healthcare worker simply checks and verifies whether the person is in fact still away. If this is the case, then the med pass is complete for that user. Preferably, the allergy information is permanently on display for a given patient as indicated at 112 by way of example. Furthermore, preferably any general comments 114 are also permanently displayed. Additional indicators “Revis” for revisit if medication was not given at first attempt and “FolUp” for follow-up of as needed medications may be provided in some embodiments as detailed below.

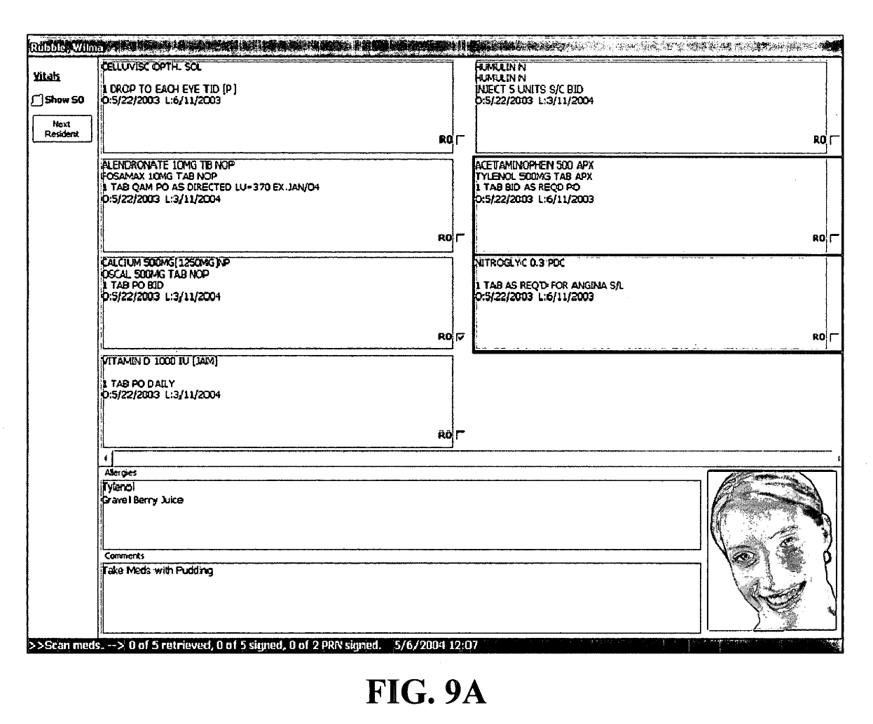

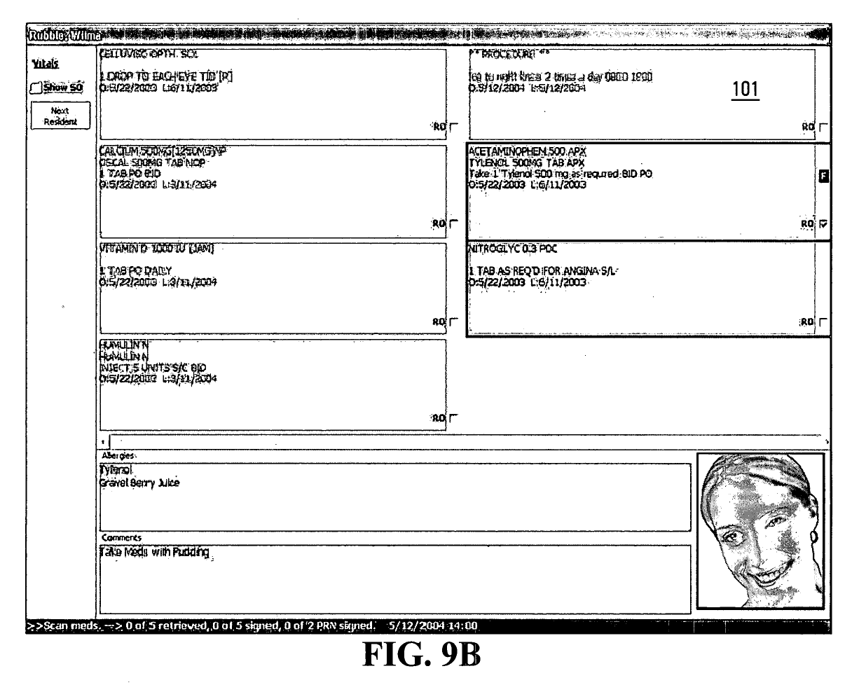

FIG. 8 shows the MAR for the patient “Wilma Rubble” selected from the list of FIG. 7. Preferably, upon displaying the residents MAR, allergy alert information is immediately and prominently displayed. An example of this is indicated at 90 in FIG. 8. In this particular example, the patient has two allergies. Before the healthcare worker can continue, preferably they are required to acknowledge the allergy alert window for example by pressing the okay button in the window 90 of the example of FIG. 8. FIG. 9A is a version of the display of FIG. 8 after the healthcare worker has cleared the allergy alert window 90.

The MAR displays all medications to be administered, and all treatments and procedures that are to be conducted. Preferably, a photo 115 is displayed (where available) when a resident's name is highlighted or the resident's bar code is scanned. Only the medications are shown for the chosen hour of administration. As needed medications may also be displayed. The example of FIG. 8 shows five medications 92,94,96,98,100 which are scheduled to be given that med pass. Two as needed medications 102,104 are also shown. These are administered at the option of the health care worker. Preferably regular medications and as needed medications are distinguished in their display, for example by displaying them using different colours. Standing orders can be added to the display by selecting 118.

Each prescription can be re-ordered at any time during the med pass by checking the RO (re-order) box. One such box is indicated at 108 of medication 92. More particularly, at any time, a healthcare worker may notice that the medication is low for the particular user and by selecting the re-order button, a re-order is generated and sent back to the pharmacy. The medication is patient specific, and a record for the particular patient and a particular medication is created and sent by the mobile terminal 38 to the application server site 13. The application server site 13 is responsible for forwarding the re-order to an appropriate pharmacy. In some embodiments the application server site 13 collects multiple orders for a given pharmacy rather than sending them one at a time.

Preferably, the re-order indication will stay checked until the ordered medication is received back from the pharmacy. This involves the application server site 13 keeping a record of the orders made for a given patient, and when the MAR information is downloaded to a portable device 38, this data will include information identifying whether or not a particular medication has been re-ordered. If it has been re-ordered, the re-order box will be checked when the patient's MAR is displayed the next time the nurse comes to see the same patient.

Preferably, if the medication is ordered again within some period of time, for example seven days, an indication will be generated to ensure that the healthcare worker in fact intends to re-order the medication.

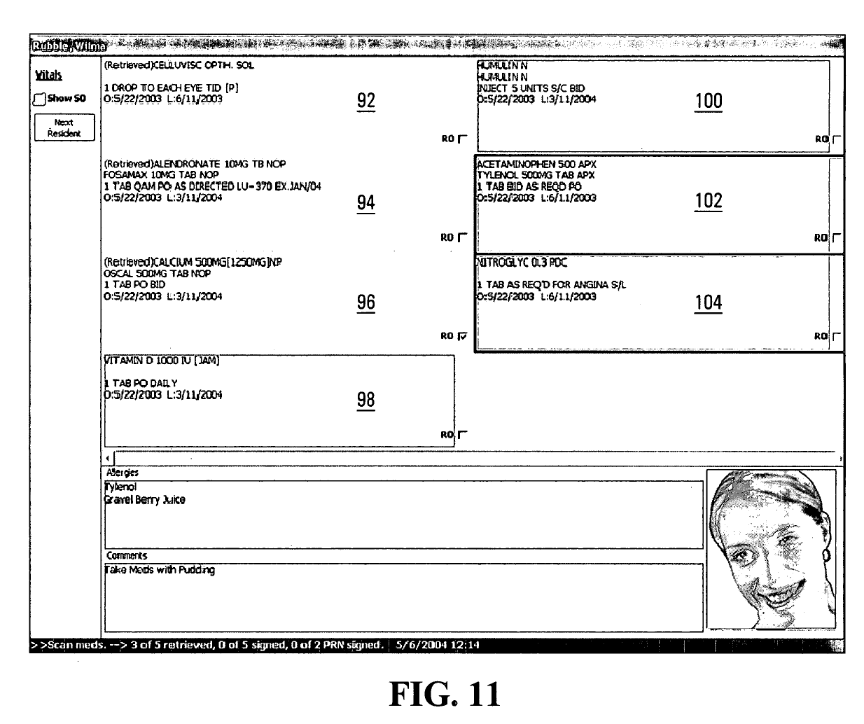

Preferably, the medications that need to be administered are presented in a colour coded manner with a first colour indicating that the medication has not yet been administered, a second colour indicating it has been retrieved, and a third colour indicating that the medication has been administered. It is not possible to show colours in this application. However, different shadings have been used instead. A first shading is shown in FIG. 8 for the medications 92,94,96,98,100 indicating that these have not yet been administered. FIG. 11 shows the same patient's MAR sometime later after having retrieved the medications of 92,94,96 as indicated by a different shading being applied for those medications.

The process of administering a medication to a patient is as follows: the healthcare worker scans the bar code of the medication; the system checks to verify whether or not this is the correct medication for that user and it is the right time and the right patient for administration. Alternate embodiments include the healthcare worker touching the medication on the screen or clicking a mouse. Assuming the medication is the correct one for administering to that patient, preferably the screen will change colour at that time indicating that the medication is ready to be given, i.e. has been retrieved. After the healthcare worker administers the drug, the healthcare worker returns and makes an entry to indicate that the medication has been administered. This might for example involve simply tapping the screen for the particular medication. Preferably at that point the colour of the display changes again to indicate that the medication has been delivered. The sequence of colours in a preferred embodiment changes from green to yellow when the medication is retrieved, and then changes from yellow to blue after the administration of the medication. Of course other different colours or different graphical distinctions may alternatively be employed to the same effect.

For a procedure or treatment, preferably once the procedure or treatment has been conducted, the healthcare worker can again indicate this on the user interface, for example by tapping the screen for the procedure or treatment. FIG. 9B shows an example of a display indicating an entry for a procedure at 101.

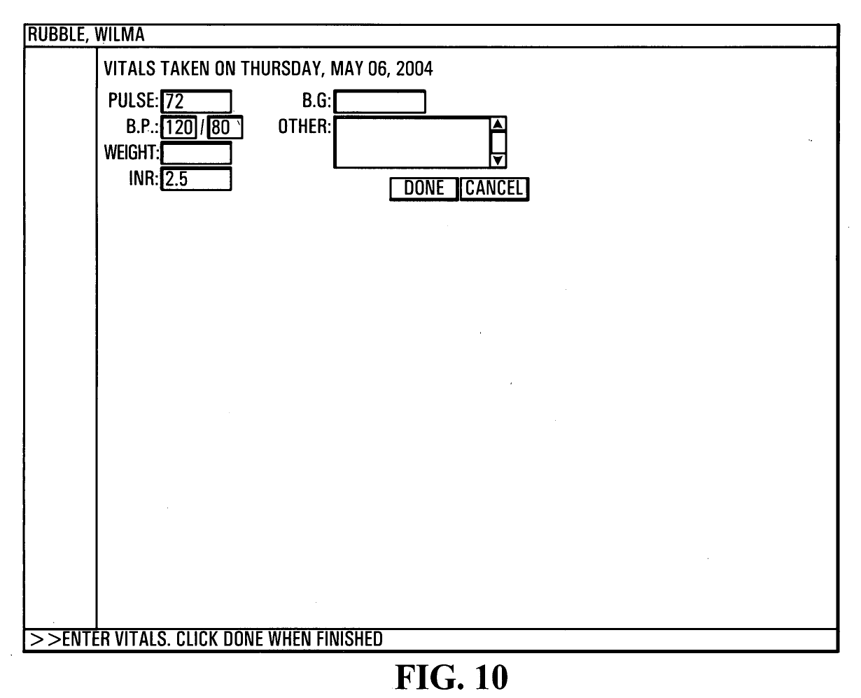

The MAR application also allows for the entry of vital sign information. In the illustrated example, this feature is accessed by selecting the vitals option 116 shown in FIG. 8. An example of a graphical user interface screen for entering a patient's vitals is shown in FIG. 10. Once a healthcare worker enters this information for a given patient, the information will be sent back to the application server site 13 once the med pass is complete.

For some medications, it may be a requirement that vitals be taken. When the healthcare worker selects that medication for administration, the pop up screen requiring the entry of vitals will then appear.

With further reference to FIG. 8, when the PRN/SO tab 84 is selected, a list of patients that are not scheduled for the current med pass is presented. For each patient, a display like that of FIG. 8 is presented upon selection of the patient, but the display will only include the as needed medications. This will allow as needed medications to be provided even though a patient is not scheduled for any particular medication. As needed medications are prescribed on a per patient basis. The standing orders appropriate for a relevant physician are displayed. In some embodiments, standing orders are displayed automatically. In some embodiments, the display of standing orders can be suppressed to reduce the amount of data required to be displayed on the portable device. This is the case in the illustrated embodiment which requires selection of “Show SO” link 118 as described above. Typically a given physician will have a set of standing orders for all residents that might require for example a particular medication be administered for headaches and some other medication be administered for diarrhoea, etc. Different physicians may have their own set of standing orders.

A “FolUp” indicator next to a name in the list of patients for a med pass means that during a previous med pass, an “as needed” or standing order medication was administered to the patient. During this med pass, the healthcare worker is required to follow-up by checking how effective the as needed medication was. A graphical user interface display screen is described below providing further details of this follow-up procedure. Preferably, these medications appear in a similar manner to regular medications in a MAR, but are displayed in a manner that distinguished them to only require follow-up. For example, they can displayed with an indicator “F” to indicate follow-up.

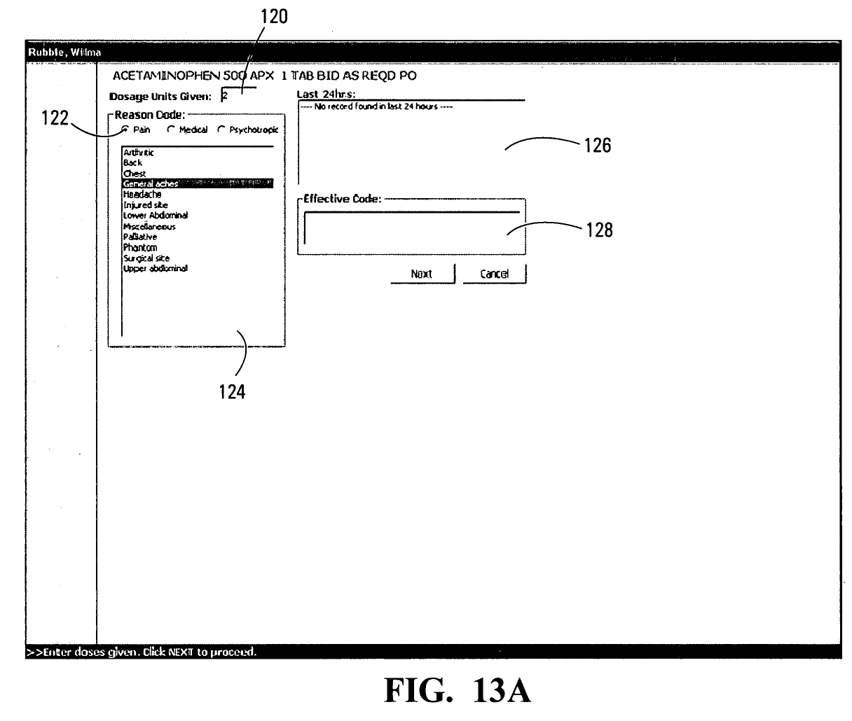

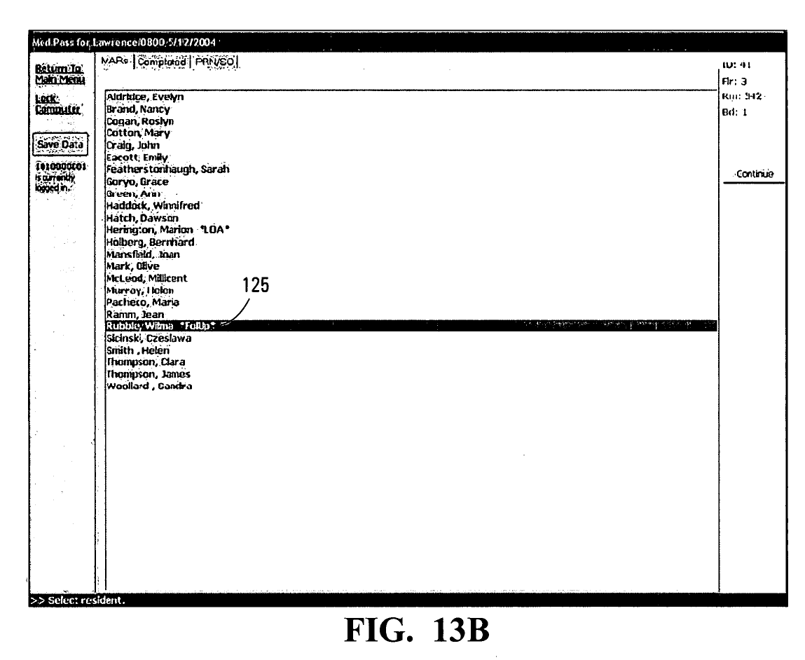

When an as needed medication is administered, a display such is shown in FIG. 13A is generated in respect of the administered medication. This allows the healthcare worker to indicate the number of dosage units given 120, and has a space for entering a reason code 122 which in the illustrated example might be a pain, medical or psychotropic reason. Each such type of reason has its own respective pull down menu with an example of a pull down menu for the pain reason code being indicated at 124. Preferably, a history is also maintained and displayed at 126 so that the healthcare worker can see how often in the recent past the same as needed medication was administered. Finally, there is a window 128 for entering an effectiveness code. This is entered in respect of a previously administered as needed medication during a previous med pass. Patients who are administered an as needed medication will be listed in the list of patients with a “FolUp” indicator next to their name. The history window 126 might for example include the doses that have been given in the last 24 hours. The patient will appear in the list of patients when follow-up is required whether or not they are to receive any other medications or treatments. An example of a display with a user having a “FolUp” indicator 125 is provided in FIG. 13B.

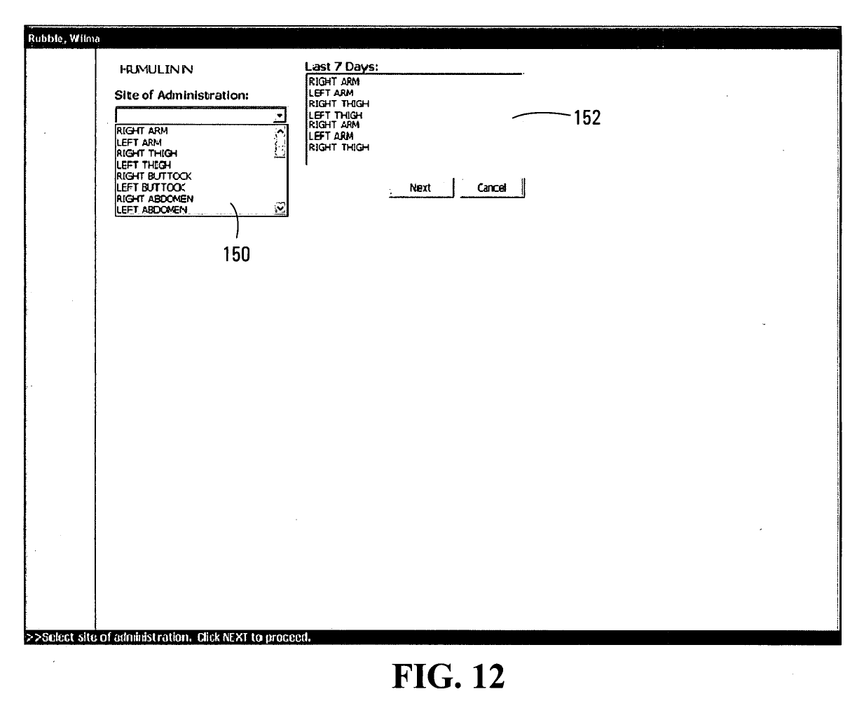

FIG. 12 is an example of a graphical user interface display for entering information on medications requiring injection. For a particular medication, there is an available list of sites for administration 150, and there is a history 152 listing the sequence of sites that were used to administer the particular medication over some period of time, for example seven days as shown. Preferably these are configurable on a per prescription basis, or per point of care facility basis.

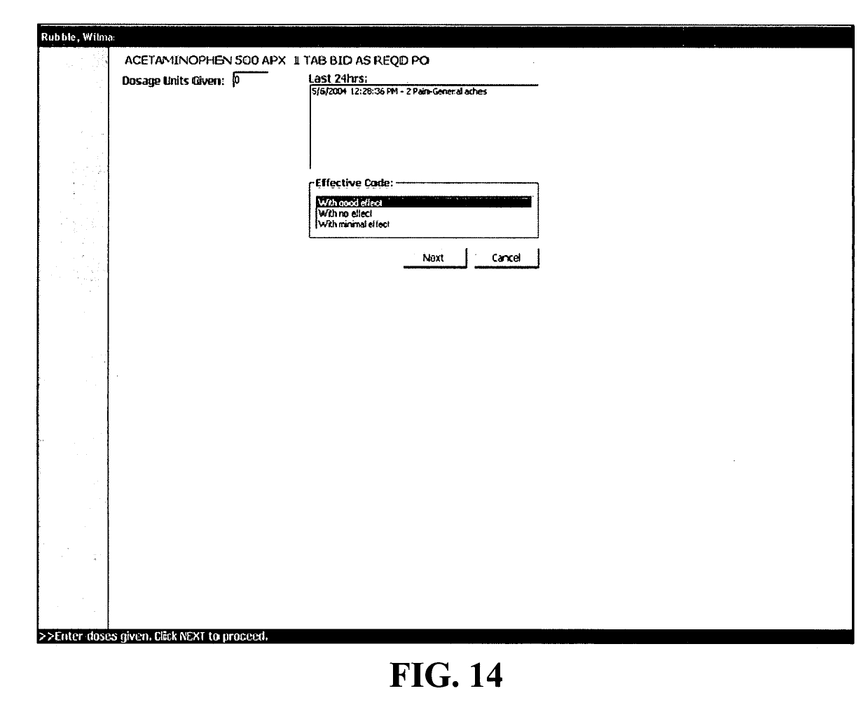

FIG. 14 shows a version of the display of FIG. 13A at a later time when follow-up on the administration of an as-needed medication is to take place.

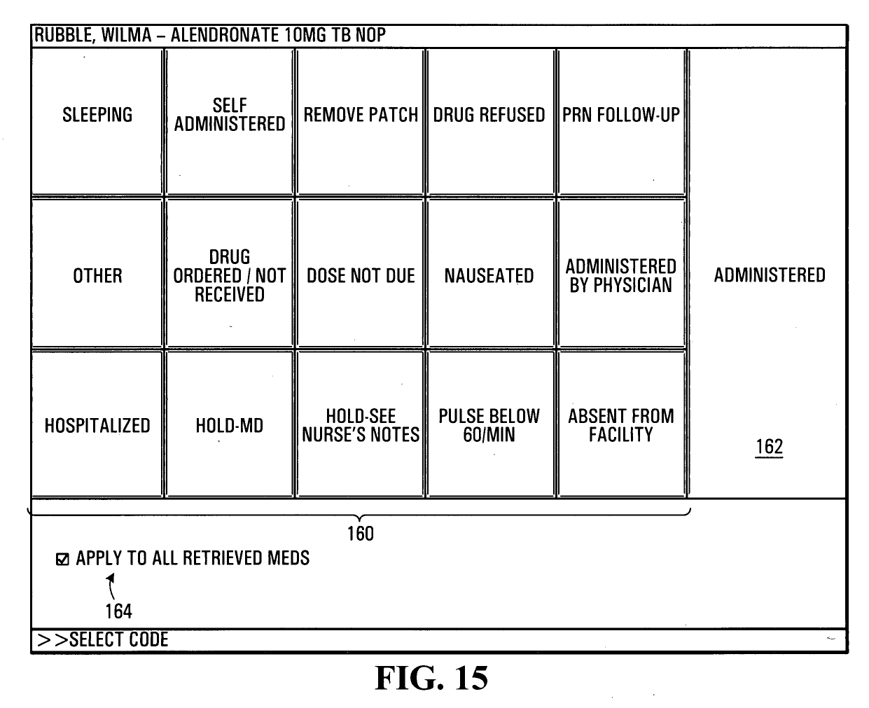

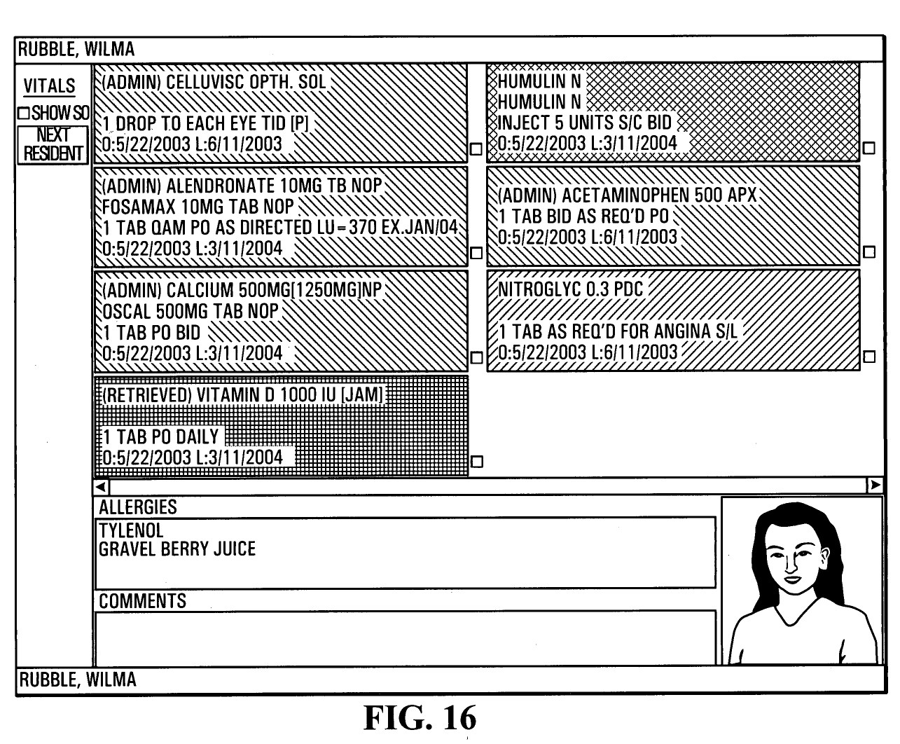

After administering or attempting to administer a drug, a screen is provided for the user to note if and how the drug was administered. The example of FIG. 15 shows a set of 15 options to indicate why a particular medication was not administered, these being generally indicated at 160, and one box for selecting that the medication was administered, as indicated at 162. One of the options 160,162 can be applied on a per medication basis, or alternatively a given selection can be applied to all of the retrieved medications by selecting box 164. Preferably, the list of options is configurable on a per point of care facility basis. Once a drug has been administered (or not administered as the case may be), the MAR display is updated and colour changed in a manner similar to that discussed above in connection with FIG. 11. FIG. 16 is a version of FIG. 11 showing now that the first three medications have been successfully administered. This is indicated by changing the colour of the display for the particular medications.

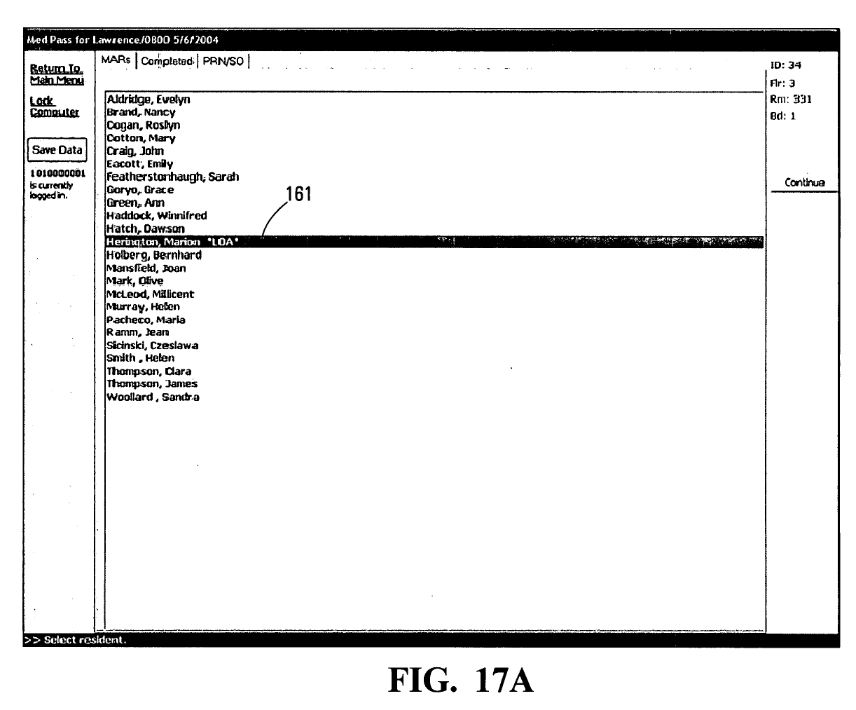

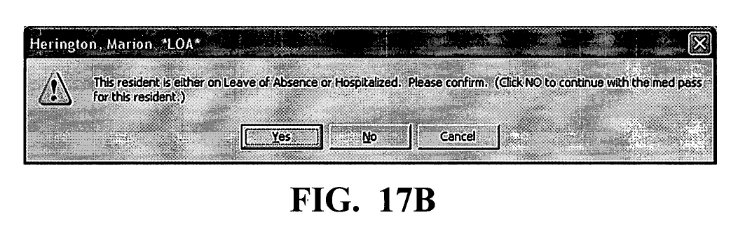

Once the administration of medications, procedures, etc. for a given patient have been completed, the display reverts to a display similar to that of FIG. 7 but with the patient to whom medications have been administered being removed from the list. An example of such a display is shown in FIG. 17A. In this particular example, the next patient on the list is indicated as being on leave of absence by the inclusion of the “LOA” indicator 161. As indicated previously, a healthcare worker should then confirm that the patient is indeed on leave of absence by pressing a “yes” in the alert window as shown in FIG. 17B and then pressing “continue” to delete the resident from the list and proceed to the next resident.

Preferably, if the healthcare worker fails to administer all of the medications to a given patient, the particular patient is not removed from the list of patients. Rather, their name is maintained on the list to indicate that a re-visit should be conducted. A “REVIS” or similar flag may be added in some embodiments to indicate that a re-visit should be conducted. In some embodiments, after an administration code 160 is entered (see FIG. 15) a revisit flag is generated only for particular codes, e.g. sleeping. A revisit flag shows up if the resident may take the medication later in that medication pass. It reminds the healthcare worker to go back and see if the resident will take the med later in the medication pass. The resident still may not take the medication and the original code would be acceptable, however if the resident takes the med on the second attempt the code would be changed to administered. More generally, the residents name is displayed in a manner indicating to revisit.

Preferably, all of the information/actions etc. obtained in the process of conducting a med pass are continuously updated in the database 17 in the application server site 13. Alternatively, the application server site 13 can be updated once at the end of a med pass.

Receive Medications Orders

The next option of FIG. 4 to be described is the “receive medications orders” tab 52 which facilitates the receipt of incoming medications.

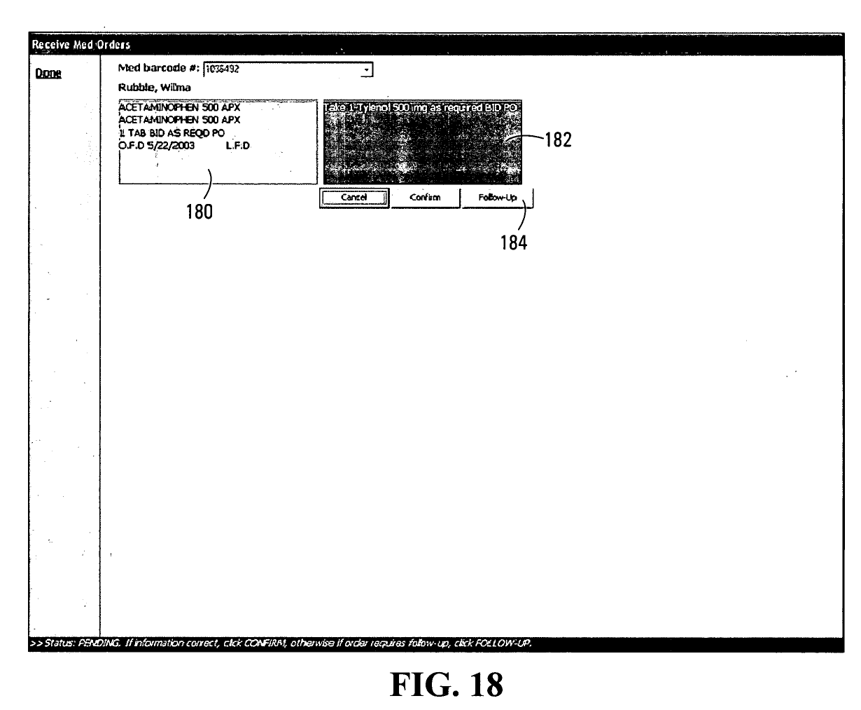

In order to receive incoming orders from the pharmacy, the user clicks “Receive Med Orders” 52 in the GUI (graphical user interface) of FIG. 4, in response to which the system downloads pending orders from the pharmacy system 11 (FIG. 18). Once the pending orders have been downloaded, the user scans the medication that has been received which results in information concerning the prescription being displayed beside the physician's order. An example of such a display is shown in FIG. 18. The pharmacy information is shown on the left 180 and the physician's order on the right 182 in FIG. 18. The physician's order can be entered into the system through the physicians' order screen at a workstation 34 of FIG. 2 or through checking the RO box as discussed with reference to FIGS. 8 and 9. The pharmacy information is then compared to the physician's order for accuracy and if correction or revision to the order is required the user clicks the “Follow-up” button 184 otherwise, the user confirms receipt with a user interface action and scans the next medication bar code. Medication receiving may also be completed in bulk by scanning a bar code on the tote or delivery box received from the pharmacy. This bar code will then receive all medications within the box.

Count Medications for Destruction or Credit

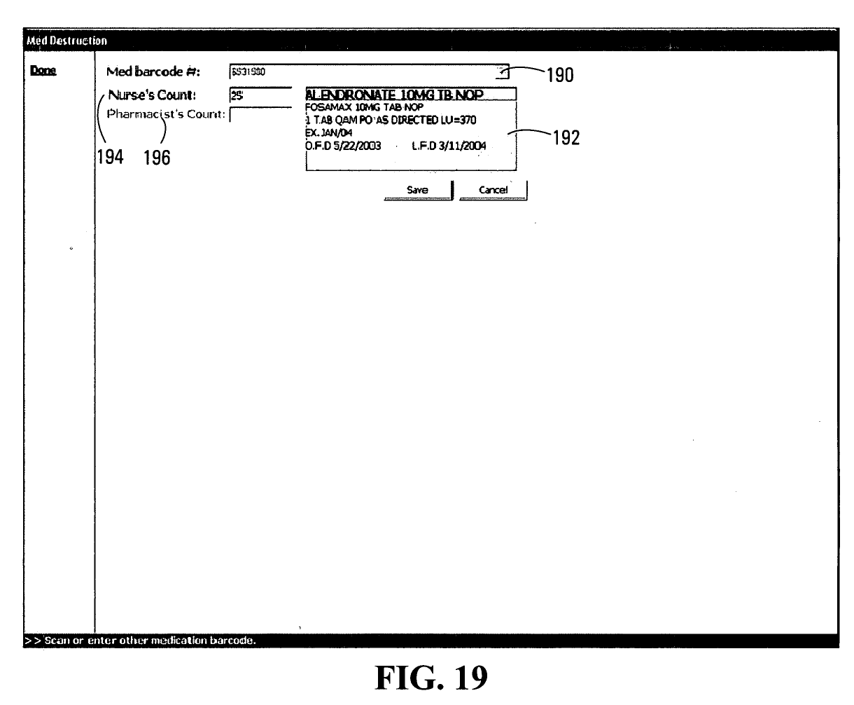

By selecting the “Count Medications for Destruction” option 54 in the screen of FIG. 4, the system downloads discontinued medications for cataloguing. Discontinued medications may for example be entered into the system via the administrative and reporting application. Once the discontinued medications have been downloaded, a screen is provided, such as shown by way of example in FIG. 19, for entering the prescription information 190 (i.e. by scanning the medication bar code), which results in the MAR information being displayed 192, entering the appropriate count of dosages remaining 194, and saving the information for later reporting. It may be possible to have a double verification of the count made. A box for entering a second count is indicated at 196. This may be conducted by the pharmacist or it may simply be a second count by the same nurse or healthcare provider. A report is then generated to calculate the proper credit to be applied to the facility.

Administrative and Reporting Application

As discussed above with reference to FIG. 2, administrative functions may be performed via workstations 34 using a administrative and reporting application within the health care facility 15 and/or in some embodiments using the portable devices 38. Preferably, the administrative function is delivered entirely in the form of web pages downloaded from the application server site 13. The functions which may be included in this administration function include patient admission, ordering or discontinuing medications, updating resident status and MARs, among others described below. Different implementations may not include all of the features. Also, as was the case for the mobile terminal described above, while specific user interface screens are presented for the purpose of illustration, it is to be clearly understood that a person skilled in the art would be able to implement the user interface in many different ways using the teachings of the invention, and within the scope of the invention.

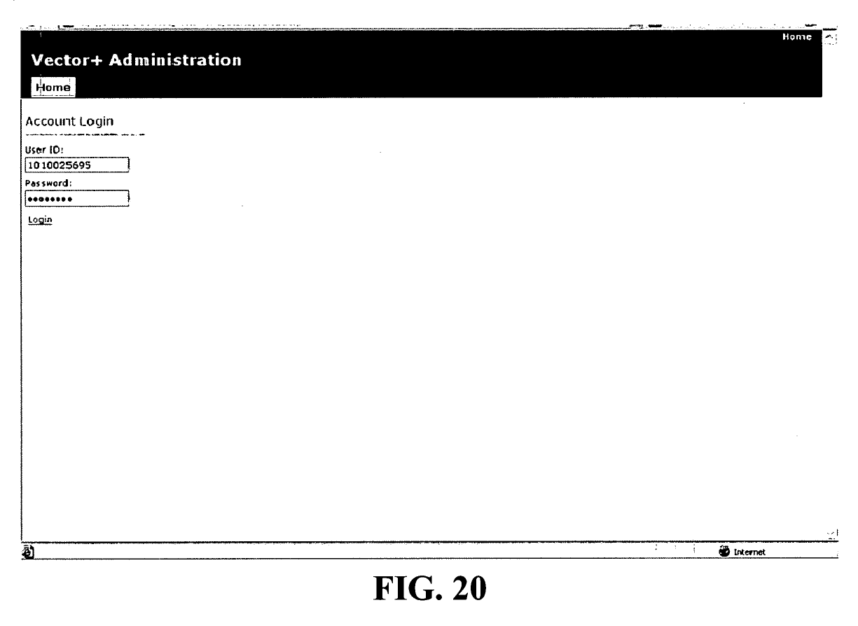

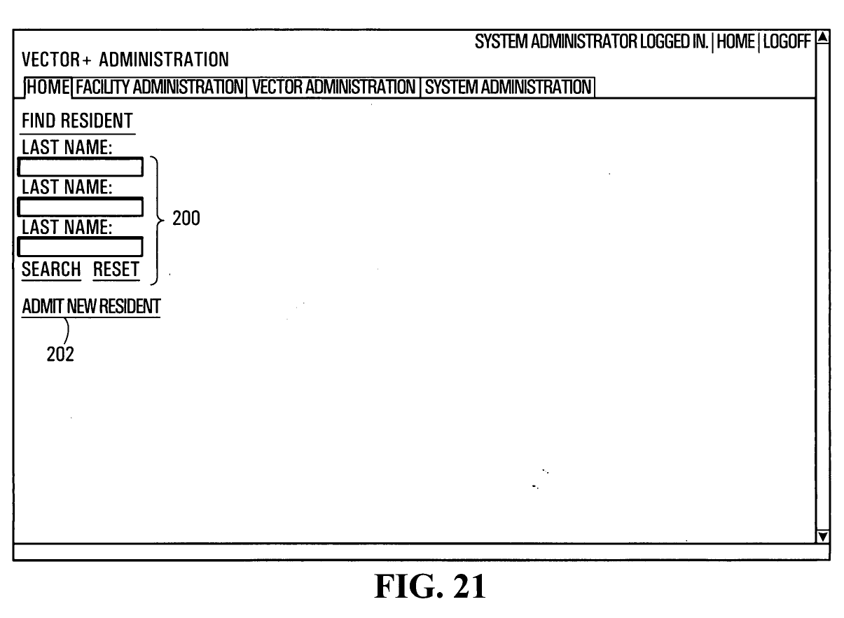

As with the mobile terminals 38, preferably, in order to use the administrative and reporting application a health care provider is required to first log in to the system at the workstation 34 by either swiping an ID card with a bar code reader or entering a user ID and PIN into the graphical user interface (GUI). An example login screen is shown in FIG. 20. The user is then presented with a Main Menu of administrative options and reports such as illustrated in FIG. 21.

Preferably, on most of the screens, an option to search for a particular resident's file is available, such as illustrated by way of example in FIG. 21. Using this screen, a user may enter one or more of the patient's health card number, last name and first name via the GUI of FIG. 21 into the fields indicated at 200. There is also the option of admitting a new resident 202.

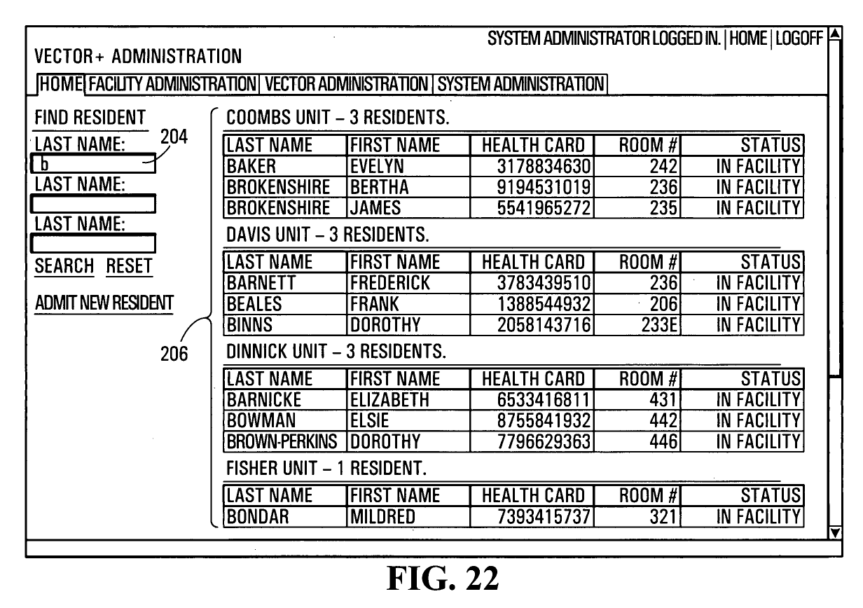

The results of the search conducted using the screen of FIG. 21 are then displayed, for example as shown in FIG. 22. In the illustrated example, the user has entered “b” in the last name field 204, and all of the residents having a last name starting with “b” are presented in FIG. 22 at 206. In the illustrated example, the last name, first name, health card number, room number, and status are displayed. Other fields may alternatively be displayed.

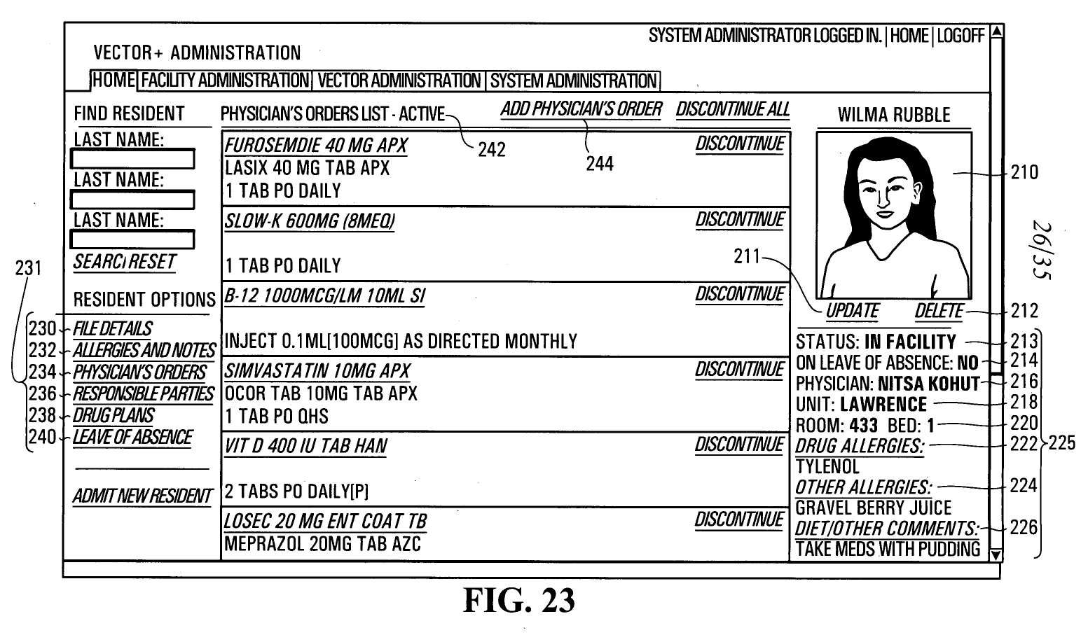

A user may then select one of the patients found in the search. With reference to FIG. 23, in response to such a selection, the system displays 210 the most recent photo of the patient. Preferably, the system allows the user to update 211 or delete 212 the current photo of the patient. Because patients' appearances may change rapidly following their admission, the ability to quickly and easily update their photos is significant. Various patient information for the patient is also presented as generally indicated at 225. In the illustrated example, this includes their status 213 indicating whether or not they are in the facility at the time; on leave of absence 214 indicating whether or not they are on leave of absence; their physician's name 216, their unit 218; their room number and bed number 220; any drug allergies 222; any other allergies 224; and any diet/other comments 226. Of course, a given implementation may include different patient information.

Having selected a given patient, a number of options are available generally indicated at 231. In the illustrated example, these consist of obtaining file details 230, allergies and notes 232, physician's orders 234, responsible parties 236, drug plans 238, and leave of absence 240. In the illustrated example, these options 231 are listed on the left hand side of the screen, the patient details 225 are listed on the right hand of the screen. Once a particular selection from the options 231 is made, the relevant information is presented in the middle of the screen. This allows for a very convenient navigation between patients and options for patients. It is noted that this is only one way to present these options and this information and that more generally, any graphical display allowing this functionality to be delivered is contemplated.

In the particular example of FIG. 23, the physician's orders 234 option has been selected, and the physician's orders list for the selected patient is indicated at 242. There is also an option for adding a new physician's order at 244. The physician's orders list lists all of the active physicians orders for the selected patient. This includes medications and treatments. Preferably, the list also includes discontinued medications. These might be listed at the bottom for example. Preferably, these discontinued medications are kept for some predetermined period of time, for example two years.

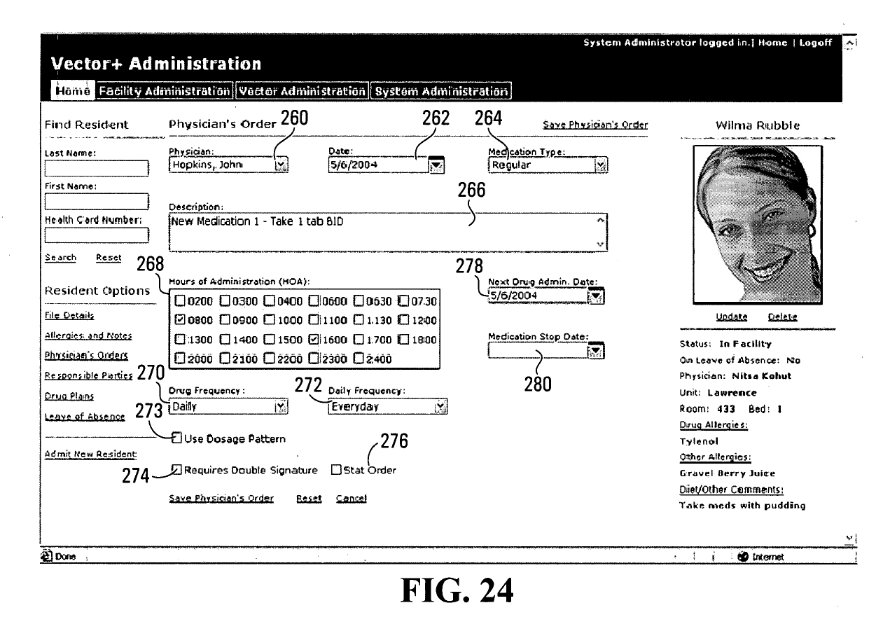

A user can select one of the displayed orders to view the details of, change date etc. of the order. An example of a physician's order details page is shown in FIG. 24. This can be used to add a new order or to amend an existing order.

There is a field for the physician 260, date 262, and medication type 264. For these fields, and many of the fields described herein, preferably a given point of care facility can customize the particular options. For example, for the medication type, these might include regular, patch, injectable, treatment, or others that might be specific to a particular facility. The description of the medication is entered at 266. The hours of administration are selected at 268. Preferably, these are customizable for a given facility. The drug frequency is entered a 270. This might be daily, weekly, or monthly for example. Depending on the frequency selected, preferably further details can be entered. For the daily frequency example shown, further details of the daily frequency can be entered at 272. For the weekly frequency, preferably, a different further details window 272 is presented which allows a selection of a particular day or days of the week. Similar options are presented for the monthly frequency.

In some embodiments, a dosage pattern can be entered, for example as indicated at 273. This allows the user to enter a sequence of doses to be administered over time. For example, the dosage pattern might be 1, 2, 3, 4, 4, 4, 4 . . . etc.

In some embodiments, a double signature can be required to enter a particular medication, as indicated at 274. This requirement is configured for a given user. In some embodiments, the medication entered is checked against a set drug or treatment database to verify whether the medication requires a double signature. A one time order can be selected by checking the “Stat Order” selection 276.

There is a field for entering the next drug administration date 278. This might be the date to start a new medication for example. There is also a field for entering a medication stop date 280. After that date, the order becomes a discontinued order.

Upon completion of a form such as that of FIG. 24. the system automatically generates an order and sends this to the appropriate pharmacy. Also, an entry is entered in the MAR for the particular patient. However, since the medication is not yet delivered, it might be displayed in a manner to distinguish it from a medication actually available for delivery, for example by displaying is as a “Temp Med”. When the medication arrives, the record becomes a normal record in the MAR. The system may also automatically generate a data transfer to other systems for usage in a care plan.

Preferably, the screen of FIG. 24 links up to a publicly available drug listing database from which all available drugs can be selected. In this embodiment, the system checks the medication entered against valid medications, dosages, recommended dosage levels, and interactions with other medications. Such checks could also continue to be completed at the pharmacy, which is the current method.

The input of new or changed physician orders can be communicated with the pharmacy quickly, and the MAR can be updated immediately. This virtually eliminates errors due to transcription and hand writing issues. In some embodiments, access to such a screen can also be given externally from the point of care facility, for example to doctors in their offices from where they can then view and update orders or approve the order using username and password authentication.

In some embodiments, a separate medication review module is provided which allows a doctor to do a medication review from his or her office electronically. The screen shows all residents due for a medication review and all necessary information. A consultant pharmacist is able to enter comments for each resident or medication prior to the review.

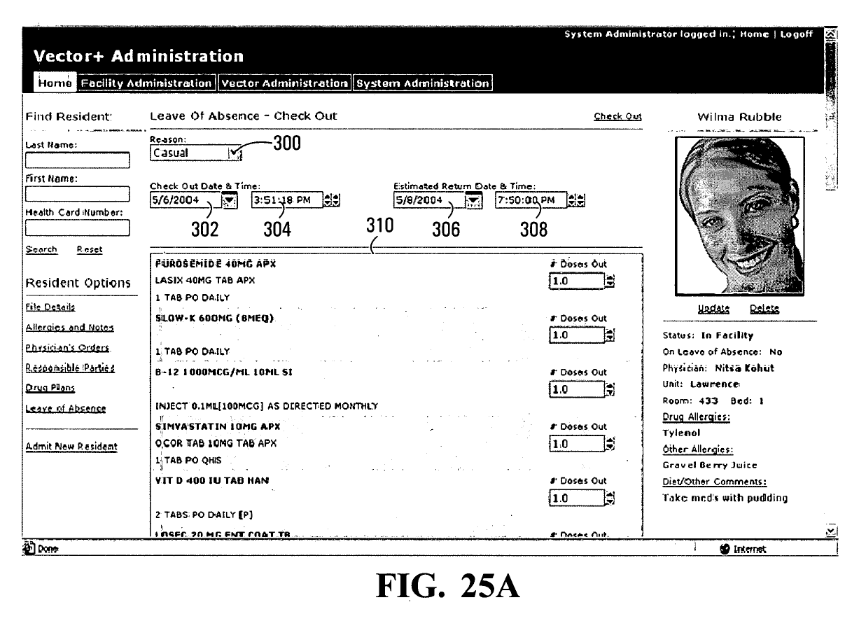

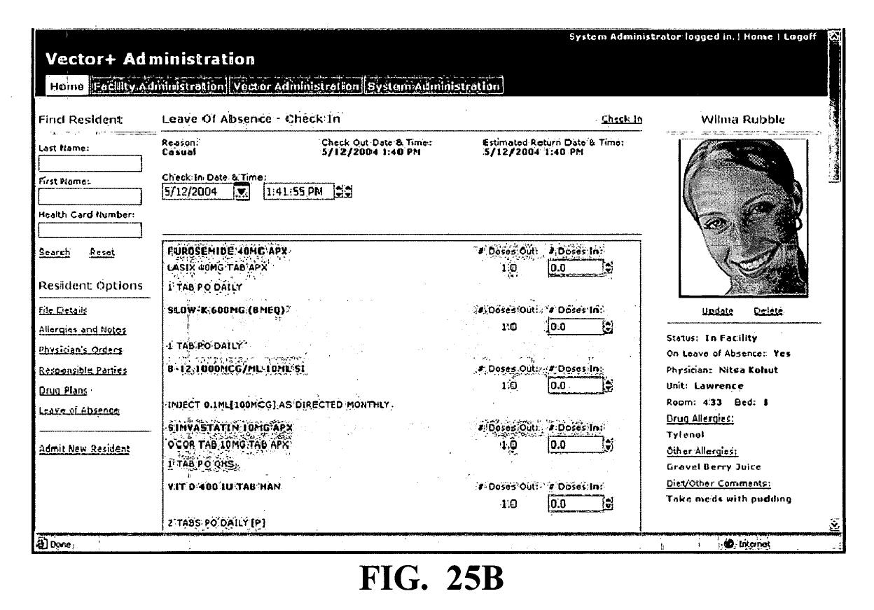

In some embodiments, functionality for a caregiver can record all of the medication needs of a resident when taking them out of the facility (LOA) is provided. An example of this is shown in FIG. 25A which is a leave of absence screen. This page has a field 300 for entering the reason for the leave of absence. There are fields for date 302 and time 304 of checkout, and estimated date 306 and time 308 of return. The complete list of medications the patient is taking is presented, and a number of doses taken out can be entered. In another embodiment, the quantity of medications to be taken out are calculated. Preferably, upon return, a doses in field is similarly entered in a check-in screen such as shown by way of example in FIG. 25B. By computing the difference between the doses out and doses in, a determination of whether the proper amount of medication was administered can be made. From this screen a complete hard copy of the MAR for the patient can be generated. This will indicate all of the medications and treatments, the times and details of application. Preferably, a release form is also printable from this page so that the responsible party can accept responsibility for the patient in writing.

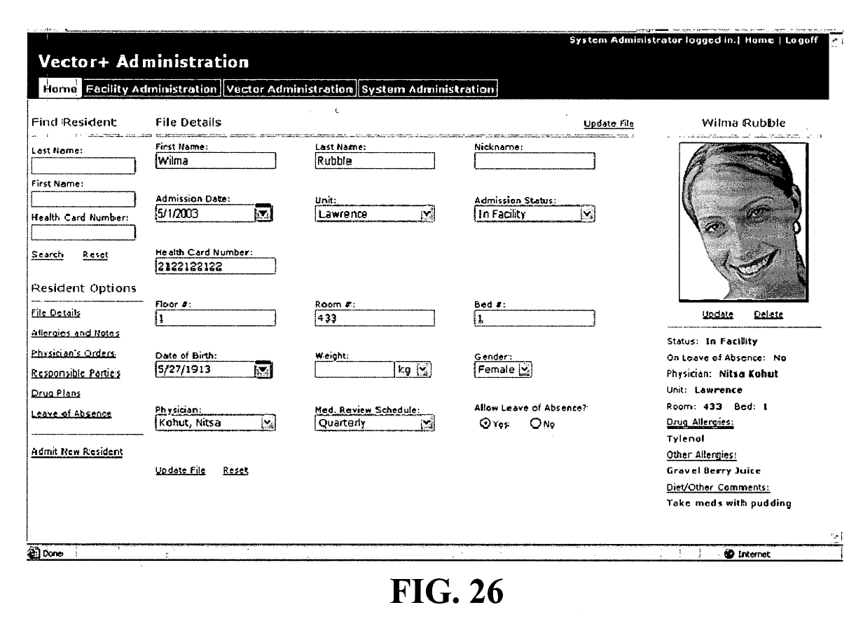

The resident's complete file (i.e. MAR) may be entered upon admission, or later revised using the GUI of FIG. 26 which shows details of the “File Details” option. In the particular example, the file details include first name, last name, nickname, admission date, unit, admission status, health card number, floor number, room number and bed number, date of birth, weight, gender, physician, med review schedule, and whether or not to allow leave of absence for the particular patient. Preferably, this information can be electronically transferred to the pharmacy's software. The information may also be electronically transferred from other clinical software used by the facility.

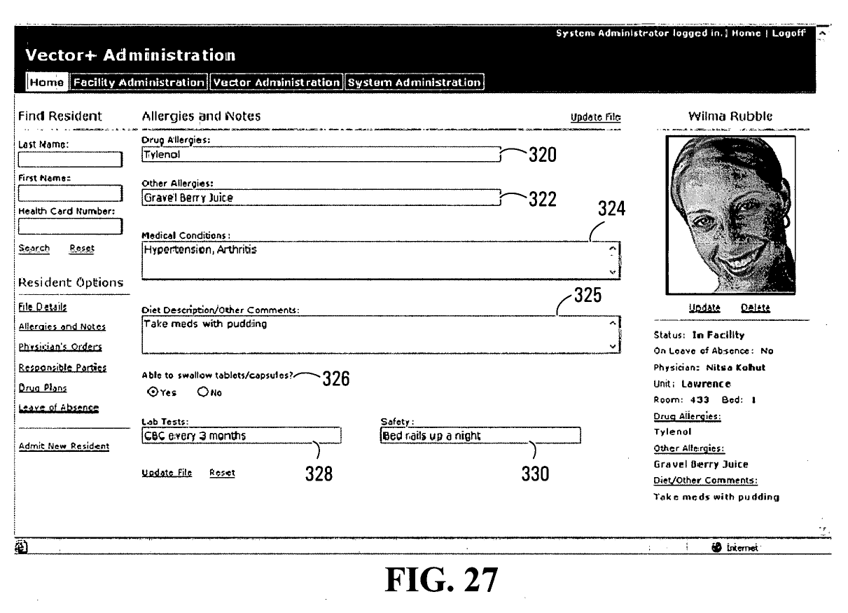

FIG. 27 shows an example of a screen for entering allergies and notes for a given resident. In this example, there are fields for entering drug allergies 320, other allergies 322, medical conditions 324, diet description/other comments 325, whether or not they are able to swallow tablets/capsules 326, how often and which type of lab tests to be performed 328, and safety notes 330. This information would typically be entered at the time of admission.

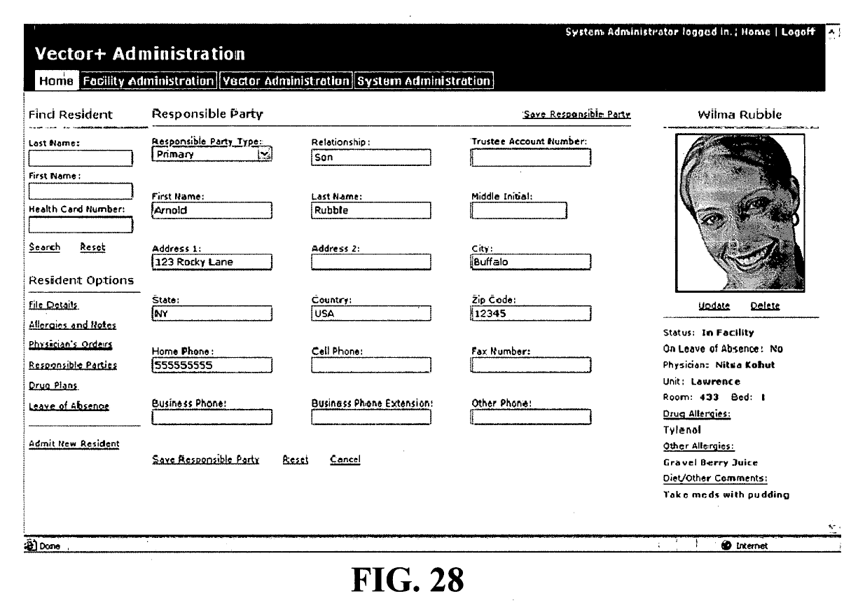

FIG. 28 is an example of a screen for entering responsible party information. Responsible parties are individuals who are allowed to take a given resident out of the facility. There may be multiple responsible parties. This screen simply allows the entry of a set of details for each responsible party.

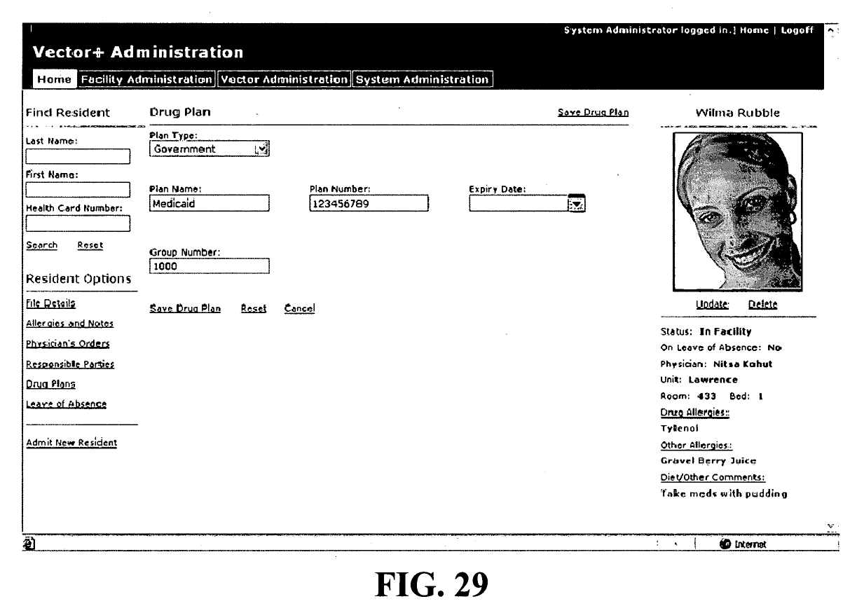

FIG. 29 shows a screen for entering drug plan information for a given resident. Preferably, multiple plans can be entered for a given patient. This information is sent to the pharmacy when a medication order is placed. In some embodiments, there may be an addition field for entering payment information for co-payment collection. This might be credit card information for example.

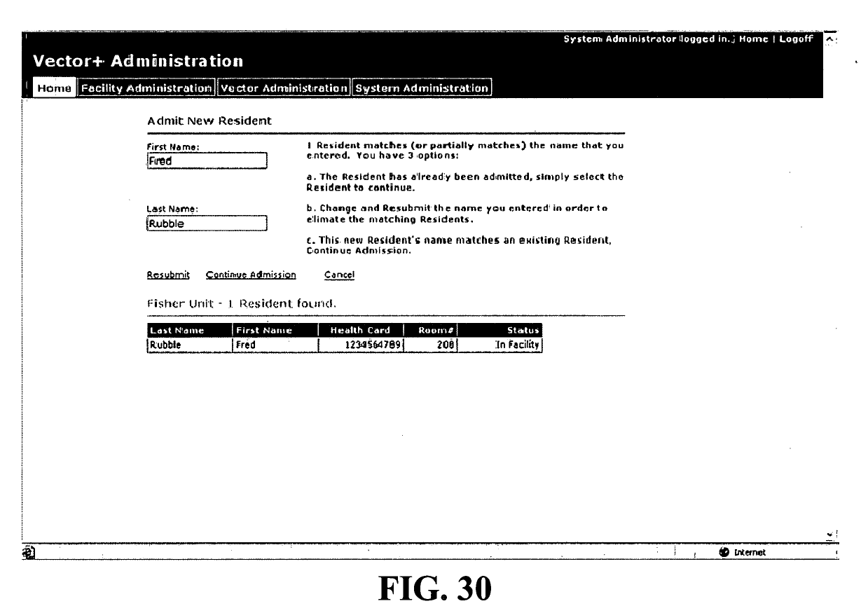

FIG. 30 shows a screen for admitting a new resident. This might for example be accessed by selecting “Admit New Resident” 202 from the screen shown in FIG. 21. This option appears in multiple screens in the illustrated embodiment. New residents are admitted by first and last name. The system searches to see if the resident has already been admitted so as to avoid duplication. If there is a second patient with the same name, then the user can continue with the admission notwithstanding this.

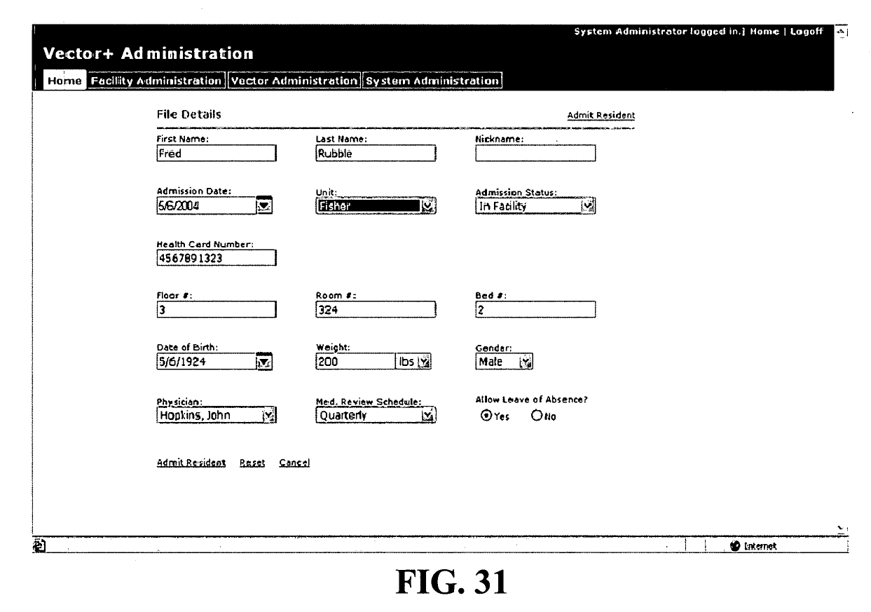

Further details of the admission process are shown in FIG. 31. This screen is for the most part the same as FIG. 26 and will not be described further.

Variations and modifications of the invention are contemplated. For example, a web server may also be provided within application server site 13 for providing administration screens to LTC administrators and servicing HTTP requests from the portable devices 38 to the master SQL server database. A separate active directory server may also be provided as a repository for user profile information, passwords, permissions, etc. Furthermore, an integration server may also be provided within application server site 13 (using XML (HL7/CHA) standards) to store med orders and prescription information submitted to and from the pharmacy systems 11. This and all other such alternative embodiments are believed to fall within the sphere and scope of the invention as defined by the appended claims. The following is a specific example of a set of tables that may be implemented in the application server site 13. It is to be understood different embodiments may use fewer tables if not all functions are implemented. Also, the particular breakdown of storage into these tables is but one example of how the information can be stored.

Titles of each Table

1. change_reason_codes: Reasons for changing a MAR code
2. dest_reason_codes: Reasons for destroying or discontinuing meds
3. drug_destruction: Collects data for counting discontinued meds
4. drug_plan: Resident drug plan information
5. drug_plan_codes: Drug plan type
6. fac_group_codes: Describes the type of facility independent, not for profit, etc.)
7. fac_hoa: Hours of administration available for a facility
8. fac_unit: Describes units within a facility
9. fac_user: Describes users of the system and what they are authorized to do
10. facility: Describes the long term care facility
11. injection_site: Describes the available sites of administration for a facility
12. leave_of_absence: Keeps history of a residents leave of absence
13. loa_med: Keeps history of medications a resident took on a leave of absence
14. loa_reason_codes: Describes the available reasons for taking a leave of absence for a particular facility
15. mar_codes: Describes available MAR codes for a facility
16. measurement: Describes the unit of measures a facility uses for vitals
17. med_detail_status_change: Tracks when the detail within medication order is changed
18. med_hoa: Tracks the hours of administration for a particular order
19. med_order_status_change: Tracks when the status of a particular order is changed
20. med_status_codes: The status available for an order (active, discontinued, pending, etc.)
21. med_type_codes: Describes the type of medication being ordered (as needed, regular, treatment, etc.)
22. medication_pharmacist_order: Tracks all history of all orders, reorders and discontinuations on all orders
23. medication_physician_order: Describes all orders available to a resident
24. medpass_change: Tracks history of all changes to a medication administration code
25. medpass_log: A log of all medication administrations that have been done
26. physician: Describes physicians available to order meds for a facility
27. prn_effective_codes: Effective codes for as needed medications
28. prn_reason_codes: Describes reasons for giving an as needed medication
29. res_med_review_schedule: How often a doctor must do medication reviews for His/Her residents
30. res_status_codes: Resident status codes (in facility, leave of absence, deceased, etc.)
31. resident: Information on individual residents within a facility
32. resp_party_codes: Describes the type of responsible party linked to a resident (primary, power of attorney, etc.)
33. responsible_party: Demographics of responsible parties
34. standing_order: Describes list of doctors standard as needed orders for all of His/Her residents
35. standing_order_restriction: Tracks any acceptions to a doctors standard orders if a resident is allergic or unable to take one of the standard orders
36. user_security: Authorizes particular user types to do differet functions within the system
37. user_types: Describes user type (registered nurse, doctor, administrator, etc.)
38. vitals: Tracks history of vitals taken for a resident

The above-described embodiments have provided a system architecture for managed delivery of medications, treatments, procedures and care. Such an embodiment features the central application server site, and the plurality of portable devices, possibly including network infrastructure between the central application server site 13 and the portable devices 38. Some embodiments will only apply to the delivery of medications while others will manage some combination of medications, treatments, procedures and care. Medication suppliers, such as pharmacies, are responsible for providing medication. In some embodiments, other external suppliers are interacted with in a managed fashion to provide treatments, procedures and care. Medication providers are also an example of external suppliers.

The preferred embodiment is a so-called “hub and spoke” model in which a single central server site provides managed delivery to multiple point of care facilities from multiple external suppliers, a point of care facility being synonymous with a health care facility. In another embodiment, the system and/or software is applied for a single health care facility, and a single supplier such as a pharmacy. In such an embodiment, it may be appropriate to locate the functionality of the central server site on location at the health care facility. This is a very different service model from the preferred embodiment in which a third party is providing the management for multiple health care facilities.

Another embodiment provides a computer program/combination of programs consisting of the drug inventory management application adapted to be run on a processing platform, such as an application server. The drug inventory management application may include any of the features described above. Preferably, the drug inventory management application includes functionality to provide appropriate portions of the MAR application on remotely accessing devices, and/or to provide appropriate portions of the administrative and reporting application on remotely accessing devices. In a preferred embodiment, this is done in the form of web pages.

The computer program, once installed in an infrastructure such as described in previous embodiments, will be capable of providing any combination of the services described herein. An embodiment of the invention provides such a computer program stored on a computer readable medium.

While specific graphical user interface screens have been provided, the invention is not limited to these particular screens. The underlying functionality can be implemented using many different screens. Furthermore, a given implementation may not include all of the functionality. The functionality to be included in a given embodiment is defined by the appended claims.

Numerous modifications and variations of the present invention are possible in light of the above teachings. It is therefore to be understood that within the scope of the appended claims, the invention may be practiced otherwise than as specifically described herein.

1. A system for managing administration of medications supplied by a medication supplier to a health care facility comprising:
a central application server site for maintaining medical administration records pertaining to residents in said health care facility and having a first data communication path to said medication supplier and a second data communication path to each health care facility; and
a plurality of portable devices for use in the health care facility for accessing and displaying said medical administration records at point of care of said residents and initiating order of said medications from said medication supplier via said central server site.

2. A system according to claim 1 wherein each medical administration record comprises at least an identification of any medications to be given, and when.

3. A system according to claim 1, wherein the second data communication path is implemented using a VPN (virtual private network).

4. A system according to claim 1 adapted to manage administration of medications to health care facilities which are long term care facilities.

5. A system for managing administration of medications supplied by a medication supplier to a health care facility comprising:
a central application server site for maintaining medical administration records pertaining to residents in said health care facility and having a first data communication path to said medication supplier and a second data communication path to each health care facility; and
a computer readable medium having a first application stored thereon adapted for use on portable devices for use in the health care facility for accessing and displaying said medical administration records at point of care of said residents and initiating order of medications from said medication supplier via said central server site.

6. A system according to claim 5 wherein the first application is delivered by the application server site as a series of downloadable pages to the portable devices.

7. A system according to claim 5 further comprising a computer readable medium having an administrative and reporting application stored thereon for managing the creation and amendment of the medical administration records.

8. A system according to claim 7 wherein the administrative and reporting application is delivered by the application server site as a series of downloadable pages to devices in the healthcare facilities.

9. A system according to claim 1 for managing administration of medications supplied by a plurality of medication suppliers to a plurality of health care facilities wherein:
the central application server site is adapted to maintain medical administration records pertaining to residents in each health care facility and has a respective first data communication path to each said medication supplier and a respective second data communication path to each health care facility; and
the system further comprising a plurality of portable devices for use in each health care facility for accessing and displaying said medical administration records at point of care of said residents and initiating order of said medications from an appropriate one of said medication suppliers via said central application server site.

10. A system according to claim 1 for managing administration of medications, treatments, procedures and care delivery to a plurality of health care facilities by a plurality of external providers comprising at least said medication supplier wherein:
the central application server site is adapted to maintain medical administration records pertaining to residents in each health care facility and has a respective first data communication path to each said external provider and a respective second data communication path to each health care facility; and
the system further comprising a plurality of portable devices for use in each health care facility for accessing and displaying said medical administration records at point of care of said residents and initiating order of said medications or services from an appropriate one of said external providers via said central application server site.

11. A system according to claim 10 wherein each said first path is secure, and each said second path is secure.

12. A system according to claim 10 further comprising at least one device in each healthcare facility adapted to run an administrative and reporting application for managing creation and amendment of the medical administration records.

13. A system according to claim 10, wherein the portable devices in a given health care facility are configured in a wireless LAN.

14. A system according to claim 10, wherein each said second data communication path between said central server site and said health care facility is implemented using a VPN, and wherein said pharmacy incorporates gateway software to contact said server site for uploading and downloading prescription information via staging tables.

15. A system according to claim 10 adapted to, for each healthcare facility:
maintain an association between the point of care facility and a respective medication supplier of said plurality of external providers;
receive medication orders from the first applications of the healthcare facility; and
send orders to the respective medication supplier.

16. A system according to claim 10 adapted to, for each healthcare facility, and for each of at least one resident in the point of care facility:
maintain an association between the patient and a respective medication supplier of said plurality of external providers;
receive medication orders from the first applications for the patient; and
send medication orders to the respective medication supplier.

17. A system according to claim 1 wherein:
upon request from one of said portable devices, the central server site generates a list of medical administration records for a selected time and downloads the list of medical administration records to the portable device, the list of medical administration records including portions of medical administration records of any residents of a selected set of residents that are scheduled to receive medication or treatment at the selected time, the portions of medical administration records including at least any medications or treatments to be administered at the selected time.

18. A system according to claim 10 wherein:
upon request from one of said portable devices, the central server site generates a list of medical administration records for a selected time and downloads the list of medical administration records to the portable device, the list of medical administration records including portions of medical administration records of any residents of a selected set of residents that are scheduled to receive medication or treatment at the selected time, the portions of medical administration records including at least any medications, treatments, procedures or care delivery to be administered at the selected time.

19. A system according to claim 17 wherein each downloaded medical administration record comprises:
an identification of any medications to be administered at the selected time;
an identification of any procedures to be performed at the selected time;
allergy information;
any other comments; and
a digital photograph.

20. A system according to claim 17 wherein the portable devices are adapted to generate a display pertaining to a selected resident containing the medications, procedures, allergy information, digital photograph and any other comments.

21. A system according to claim 19 further adapted to receive a user input upon retrieval of a medication and upon administration of a medication, and to display each medication in a first manner initially, in a second manner visually distinct from said first manner after the medication has been retrieved, and in a third manner visually distinct from said first and second manners after the medication has been administered.

22. A system according to claim 20 further adapted to provide a user input at the portable device for selecting reordering of a given medication;
wherein responsive to said user input at the portable device, the application server site generates an order for the medication and sends this automatically to an appropriate medication provider.

23. A system according to claim 18 further adapted to provide a user input at the portable device for selecting reordering of a given medication, treatment, procedure or care delivery;
wherein responsive to said user input at the portable device, the application server site generates an order for the medication, treatment, procedure or care delivery and sends this automatically to an external provider.

24. A system according to claim 22 further adapted to display that a given medication has been reordered until the medication has been received.

25. A system according to claim 1 further adapted to store vital sign information for residents.

26. A system according to claim 25 comprising a user interface on the portable devices for entering vital sign information.

27. A system according to claim 21 further adapted to generate a request to take vital signs prior to administering a given medication.

28. A system according to claim 19 adapted to display a list of resident identifiers of residents to receive medication, and to display each resident's identifier in a different manner after completing a med pass for the resident.

29. A system according to claim 28 wherein displaying a resident's identifier in a different manner after completing a med pass for the resident comprises listing them on a different display page on the portable device.

30. A system according to claim 17 further adapted to present a plurality of options on the portable device for each medication to indicate whether the medication was successfully administered or not, and if not to enter one of a plurality of reason codes.

31. A system according to claim 30 wherein the reason codes are customizable on a per healthcare facility basis.

32. A system according to claim 17 adapted to display residents who are on leave of absence so that a verification that they are in fact still out of the facility can be performed.

33. A system according to claim 21 wherein each said medical administration record further comprises an identification of any as needed medications for the associated resident, the system being further adapted to track administration of any as needed medications.

34. A system according to claim 21 wherein each said medical administration record further comprises an identification of any as needed medications for the associated resident, the system being further adapted to track administration of any as needed medications;
wherein the system is further adapted to initially display as needed medications in a manner visually distinct from said first manner.

35. A system according to claim 34 further adapted to track a reason for administration of an as needed medication.

36. A system according to claim 34 further adapted to maintain a history of administration of an as needed medication.

37. A system according to claim 34 further adapted to automatically include a medical administration record of a resident who was administered an as needed medication in a list of medical administration records generated at a subsequent time and request effectiveness information from a health care worker in respect of how effective the as needed medication was.

38. A system according to claim 33 further adapted to include a resident with no scheduled medications or treatments in a list of medical administration records for a selected time if they have as needed medications.

39. A system according to claim 38 adapted to include a resident with no scheduled medications or treatments in a list of medical administration records for a selected time if they have as needed medications in a manner visually distinct from residents with scheduled medications or treatments.

40. A system according to claim 34 further adapted to track standing orders for each of a plurality of health care providers.

41. A system according to claim 40 further adapted to download for each resident the standing order information pertaining to an associated one of said healthcare providers.

42. A system according to claim 21 wherein each said medical administration record further comprises an identification of standing order medications for the associated resident, the system being further adapted to track administration of any standing order medications.

43. A system according to claim 21 wherein each said medical administration record further comprises an identification of standing order medications for the associated resident, the system being further adapted to track administration of any standing order medications;
wherein the system is further adapted to initially display standing order medications in a manner visually distinct from said first manner.

44. A system according to claim 43 providing a user selection of whether or not to display standing order medications for the associated resident.

45. A system according to claim 42 further adapted to track a reason for administration of a standing order medication.

46. A system according to claim 42 further adapted to maintain a history of administration of a standing order medication.

47. A system according to claim 42 further adapted to automatically include a medical administration record of a resident who was administered a standing order medication in a list of medical administration records generated at a subsequent time and request effectiveness information from a health care worker in respect of how effective the standing order medication was.

48. A system according to claim 42 further adapted to include a resident with no scheduled medications or treatments in a list of medical administration records for a selected time if there are standing order medications.

49. A system according to claim 17 further adapted to:
after completing an attempt at administering medications to or treatments for a given resident wherein a failure to administer one or more medications or treatments has occurred, the system is adapted to keep the resident's medical administration record on the list of medical administration records to allow a re-visit during a current med pass.

50. A system according to claim 49 adapted to request a reason code from a health care worker following failure to administer a medication.

51. A system according to claim 50 adapted to allow a re-visit only for failures with particular reason codes.

52. A system according to claim 50 adapted to display a resident's identifier in a visually distinct manner if they are to receive a revisit.

53. The system of claim 1, wherein said portable devices incorporate a user log-in function to restrict access to authorized persons.

54. The system of claim 1, wherein said portable devices include a user interface for displaying selected ones of said medical administration records according to location, time and date within said health care facility.

55. The system of claim 54, wherein said selected ones of said medical administration records are accessed by database replication between said portable devices and said central server site.

56. The system of claim 54, wherein said portable devices include a user interface for displaying sites for injection administration of said drugs to said residents.

57. The system of claim 1, further comprising functions for receiving medication orders enabling comparison of a received medication and a medication order and for entering follow-up notations based on any discrepancies therebetween.

58. The system according to claim 57 adapted to:
upon user request, download outstanding medication orders to one of said portable devices;
upon scanning of a received medication, generate a display containing information pertaining to the scanned medication, and information pertaining to a medication order for the particular received medication so that discrepancies can be identified.

59. A system according to claim 1 further comprising:
at least one function for reporting destruction of medications.

60. A system according to claim 59 comprising:
a user interface on the portable device for entering an identifier of a medication to be destroyed, and for entering at least one count of the medication to be destroyed.

61. A system according to claim 60 wherein the application server site collects information pertaining to the type and number of medication destroyed and generates a report upon request for use in obtaining credit for the destroyed medications.

62. A system according to claim 1, further comprising at least one device executing an administrative and reporting application for creating and updating said medical administration records and initiating physician's orders of said medications.

63. A system according to claim 62 wherein the at least one device comprises at least one workstation.

64. A system according to claim 62 wherein the administrative and reporting application runs on the portable devices.

65. A system according to claim 62, wherein said administrative and reporting application incorporates a user log-in function to restrict access to authorized persons.

66. A system according to claim 62, wherein said administrative and reporting application includes a user interface for uploading resident photographs to said central server site.

67. A system according to claim 62, wherein said administrative and reporting application includes a user interface for updating status of said residents.

68. A system according to claim 62 wherein the administrative and reporting application comprises a plurality of screens downloadable by the application server site.

69. A system according to claim 62 wherein each physicians order comprises:
an identity of a medication and an identity of the resident the medication is being ordered for;
time of administration information; and
frequency of administration information.

70. The system according to claim 69 adapted to generate an order for the medication, and to enter the medication on the resident's medical administration record.

71. The system according to claim 69 adapted to present a list of all medications or treatments for a selected resident, and to allow amendment of time or frequency information, and to allow discontinuing a medication.

72. A system according to claim 62 further comprising:
a user interface screen(s) for indicating that a particular resident is going on leave of absence.

73. A system according to claim 72 wherein the user interface screen(s) for indicating that a particular resident is going on leave of absence lists all of the medications being administered to the resident and collect “doses out” information identifying numbers of doses of each medication being taken out of the facility.

74. A system according to claim 73 wherein the user interface screen(s) for indicating that a particular resident is going on leave of absence include an input for indicating the resident has returned, and list all of the medications being administered to the resident and collect “doses in” information identifying numbers of doses of each medication being returned to the facility.

75. A system according to claim 74 adapted to calculate an appropriate amount of medication for a period of leave of absence.

76. A system according to claim 74 further adapted to compute a difference between the doses out and the doses in and to determine whether a proper amount of medication was administered for an amount of time the particular resident was on leave of absence.

77. A system according to claim 62 wherein the administrative and reporting application is adapted to collect at least one of: allergy information, medical condition information, and any other comments, and lab test requirements.

78. A system according to claim 62 wherein the administrative and reporting application is adapted to collect responsible party information pertaining to individuals who are to be allowed to leave the facility with a given resident.

79. A system according to claim 62 wherein the administrative and reporting application is adapted to collect drug plan information for each resident and to include drug plan information in orders for medication made for the resident.

80. The system of claim 1, adapted to track a health care facility inventory of at least one of said medications.

81. The system of claim 80, further adapted to track administration of each of said at least one of said medications to said residents and to update said health care facility inventory of said at least one of said medications.

82. The system of claim 5, adapted to track a health care facility inventory of at least one of said medications.

83. The system of claim 82, further adapted to track administration of each of said at least one of said medications to said residents and to update said health care facility inventory of said at least one of said medications.

84. A computer readable medium having executable instructions stored thereon for managing administration of medications supplied by a medication supplier to a health care facility, the executable instructions comprising:
code for maintaining medical administration records pertaining to residents in said health care facility and for communicating via a first data communication path to said medication supplier and via a second data communication path to each health care facility; and
code for use on a plurality of portable devices for use in the health care facility for accessing and displaying said medical administration records at point of care of said residents and initiating order of said medications from said medication supplier via said central server site.

85. A computer readable medium according to claim 80 adapted to manage administration of medications supplied by a plurality of medication suppliers to a plurality of health care facilities, the instructions further comprising:
code for maintaining medical administration records pertaining to residents in each health care facility and communicating via a respective first data communication path to each said medication supplier and via a respective second data communication path to each health care facility; and
code for use on portable devices for use in each health care facility for accessing and displaying said medical administration records at point of care of said residents and initiating order of said medications from an appropriate one of said medication suppliers via said central application server site.

86. A computer readable medium according to claim 81 for managing administration of medications, treatments, procedures and care delivery to a plurality of health care facilities by a plurality of external providers comprising at least said medication supplier, the instructions comprising:
code for maintaining medical administration records pertaining to residents in each health care facility and communicating via a respective first data communication path to each said external provider and via a respective second data communication path to each health care facility; and
code for use on portable devices in each health care facility for accessing and displaying said medical administration records at point of care of said residents and initiating order of said medications or services from an appropriate one of said external providers via said central application server site.

87. A computer readable medium according to claim 82 further comprising:
code for an administrative and reporting application for managing creation and amendment of the medical administration records.
